(19) United States
(12) Patent Application Publication
(10) Pub. No.: US 2024/0342177 A1
(43) Pub. Date: Oct. 17, 2024

(54) METHODS OF TREATING BRAIN CANCER

(71) Applicant: Detsamma Investments Pty Ltd, Austinmer (AU)

(72) Inventors: Richard David RANSON, Austinmer (AU); Christian Damian TOOULI, Glebe (AU); David Simon ZIEGLER, Dover Heights (AU)

(21) Appl. No.: 18/294,806

(22) PCT Filed: Aug. 4, 2021
(86) PCT No.: PCT/AU2021/050848

§ 371 Date: Feb. 2, 2024

(51) Int. Cl. A61K 31/519; A61K 9/08; A61K 31/513; A61K 45/06; A61K 47/40; A61P 35/00

(52) U.S. Cl. CPC A61K 31/519; A61K 9/08; A61K 31/513; A61K 45/06; A61K 47/40; A61P 35/00

(57) ABSTRACT

Disclosed herein are pharmaceutical compositions for the treatment of cancer. In particular, disclosed herein are methods and uses of the pharmaceutical compositions for treating, preventing and/or managing cancers of the brain and/or central nervous system. The pharmaceutical compositions may comprise: (i) 5-FU, a pharmaceutically acceptable salt thereof, or a pharmaceutically acceptable analogue thereof; (ii) folinic acid, or a pharmaceutically acceptable salt thereof; and (iii) a cyclodextrin or a pharmaceutically acceptable salt thereof.

### TECHNICAL FIELD

The present disclosure relates to pharmaceutical compositions for the treatment of cancer. In particular, the present disclosure relates to methods and uses of pharmaceutical compositions which may be used for treating, preventing and/or managing cancers of the brain and/or central nervous system.

### BACKGROUND

Cancer is a common cause of death in the world; about 10 million new cases occur each year, and cancer is responsible for about 12% of deaths worldwide, making cancer the third leading cause of death.

Brain and nervous system tumours are among the deadliest of all forms of cancer. More than two-thirds of adults diagnosed with glioblastoma—the most aggressive type of brain cancer—die within two years of diagnosis. Brain cancers are also the most common and most lethal of all paediatric solid tumours. Furthermore, children with these tumours, who survive and enter adulthood, will often be affected by the long-term consequences of exposing the developing brain to medical interventions, including surgery, radiotherapy and/or chemotherapy.

Brain tumours have proved challenging to treat, due to issues such as the limiting biological characteristics and invasive growth patterns of these cancers. Various factors, such as location beyond the reach of neurosurgeons and the tight control of the blood-brain barrier, make the treatment of brain tumours arguably more problematic than other cancers. Furthermore, the unique developmental, genetic, and micro environmental features of the brain frequently render these cancers resistant to treatments.

Most neoplastic brain lesions are metastases arising from cancers outside the central nervous system, such as breast cancer and metastatic colorectal cancer (mCRC), and are up to ten times more common than primary brain tumours. Gliomas and meningiomas are the most common types of primary brain tumour.

Malignant gliomas, the most common type of primary brain tumours, are aggressive, highly invasive, and neurologically destructive tumours which are among the deadliest of all human cancers. Of the estimated 17,000 new brain tumours diagnosed each year in the United States, about half are malignant gliomas. Malignant glioma cells produce very invasive brain tumours with infiltration of both white and grey matter. At the time of diagnosis, microscopic extension through much of the neural axis by malignant glioma is the rule. Such extension by motile invading cells underlies the incurability by surgery of most gliomas, even when they appear small and restricted in nature.

Malignant gliomas, the most common adult-onset neurological neoplasms, encompass a family of primary central nervous system tumours including glioblastoma, astrocytoma, oligodendroglioma, and ependymoma, along with the juvenile onset neoplasms such as juvenile pilocystic astrocytoma. Gliomas including pilocytic astrocytomas, pleomorphic xanthoastrocytomas and ependymomas occur less frequently. In children, the most common types of glioma are pilocytic astrocytomas and diffuse midline gliomas including diffuse intrinsic pontine gliomas of various grades.

Glioblastoma (including glioblastoma multiforme, GBM), is the most serious form of malignant glioma, characterised by extremely aggressive brain tumours which generally arise in the upper brain (cerebrum), but which may also occur elsewhere in the central nervous system (CNS), such as in the spinal cord, cerebellum, brain stem, or optic chiasm. Low-grade gliomas, including astrocytomas, oligodendrogliomas, and pilocytic astrocytomas, account for about 25% of all primary brain tumours, and over time most of these low-grade tumours dedifferentiate into more malignant gliomas. Diffuse astrocytomas are predominantly located in the cerebral hemispheres of adults and have an inherent tendency to progress to anaplastic astrocytoma and (secondary) glioblastoma. The majority of glioblastomas develop de novo (primary glioblastomas), without an identifiable less-malignant precursor lesion.

Even with optimal state of the art therapy, the median survival of patients with glioblastomas is only 12 to 15 months. When these tumours recur, conventional cytotoxic therapies produce minimal benefit, with only 8 to 15% of patients alive and free from progression at 6 months. Glioblastoma is the most lethal brain tumour. Without treatment the medium survival time is 4 months, and with available treatment about 15 months. Many patients do not survive longer than 6 months from diagnosis and most die within 2 years. Only a few survive for as long as 5 years.

Despite many advances in the field of cancer treatment, including surgery, radiation, and chemotherapy, none of the available treatments have been able to significantly improve the survival rate of brain and nervous system cancers patients. Therefore, there remains an urgent need in the art for the discovery of new effective chemotherapeutic agents for therapeutic intervention with cancers of the brain and/or CNS.

Any discussion of documents, acts, materials, devices, articles or the like which has been included in the present specification is not to be taken as an admission that any or all of these matters form part of the prior art base or were common general knowledge in the field relevant to the present disclosure as it existed before the priority date of each claim of this application.

### SUMMARY

The present inventors have surprisingly discovered that a composition comprising, for example, 5-fluorouracil (5-FU), folinic acid, and a cyclodextrin compound, is able to achieve adequate concentration in the brain and to be effective in the treatment and/or prophylaxis of diseases and disorders of the brain and/or CNS. For example, the composition comprising: (i) 5-FU, a pharmaceutically acceptable salt thereof, or a pharmaceutically acceptable analogue thereof; (ii) folinic acid, or a pharmaceutically acceptable salt thereof; and (iii) a cyclodextrin or a pharmaceutically acceptable salt thereof, is able to treat diseases of the brain and nervous system cancers including, but not limited to: gliomas, glioblastomas, and ependymomas.

In a first aspect, disclosed herein is a method of treating and/or preventing a cancer of the brain and/or central nervous system in a subject in need thereof, the method comprising administering to the subject a composition comprising:
(i) 5-FU, a pharmaceutically acceptable salt thereof, or a pharmaceutically acceptable analogue thereof;
(ii) folinic acid, or a pharmaceutically acceptable salt thereof; and
(iii) a cyclodextrin or a pharmaceutically acceptable salt thereof.

In a second aspect, disclosed herein is a composition comprising:
(i) 5-FU, a pharmaceutically acceptable salt thereof, or a pharmaceutically acceptable analogue thereof;
(ii) folinic acid, or a pharmaceutically acceptable salt thereof; and
(iii) a cyclodextrin or a pharmaceutically acceptable salt thereof, for use in the treatment and/or prevention of a cancer of the brain and/or central nervous system.

In a third aspect, disclosed herein is use of a composition comprising:
(i) 5-FU, a pharmaceutically acceptable salt thereof, or a pharmaceutically acceptable analogue thereof;
(ii) folinic acid, or a pharmaceutically acceptable salt thereof; and
(iii) a cyclodextrin or a pharmaceutically acceptable salt thereof,
   in the manufacture of a medicament for the treatment and/or prevention of a cancer of the brain and/or central nervous system.

In fourth aspect, disclosed herein is a method of reducing the size of, or slowing the growth of, brain neoplasms in a subject in need of such treatment, comprising administering to the subject an effective amount of a composition comprising:
(i) 5-fluorouracil (5-FU) or an analogue or a pharmaceutically acceptable salt thereof;
(ii) folinic acid, or a pharmaceutically acceptable salt thereof; and
(iii) a cyclodextrin, or a pharmaceutically acceptable salt thereof.

With regards to the disclosed methods and use of the compositions as described herein, at least according to some embodiments or examples there may be provided one or more of the following advantages over known methods:
(i) improved delivery of both 5-FU and folinic acid by allowing simultaneous administration of the two compounds;
(ii) minimised side effects that are prevalent when 5-FU is administered at higher pH values of about 9;
(iii) reduced administration time and costs (in the clinic) by enabling the combination of an anticancer agent and its biomodulator to be administered in a single dose;
(iv) significantly simplified administration because lower pH means that a central line usually required to prevent phlebitis may not be required;
(v) further to point (iv) as a result can reduce stress on the patient;
(vi) improved efficacy associated with the concomitant delivery of, for example, 5-FU and its biomodulator folinic acid;
(vii) reduced systemic toxicity possibly due to the reduced pH of the formulation and/or the addition of a cyclodextrin;
(viii) as a result of reduced toxicity, the ability to deliver higher doses of 5-FU, the ability to deliver the drug to frailer, younger or less robust patients, and the ability to treat patients for longer periods than is possible with 5-FU administered separately; and
(ix) as a result of the pH of the compositions being closer to physiological pH, reduced risk of cardiotoxicity caused via breakdown products of 5-FU, leading to the ability to treat patients that are contraindicated for 5-FU/fluoropyrimidine treatment due to cardiovascular risks.

It will be appreciated that the embodiments of each aspect of the present disclosure may equally be applied to each other aspect, mutatis mutandis.

### BRIEF DESCRIPTION OF DRAWINGS

Whilst it will be appreciated that a variety of embodiments disclosed herein may be utilised, in the following, described herein are a number of examples with reference to the following drawings:

FIG. 1 shows a dose-tolerance relationship for escalating-dose administration of the composition used in the methods of the present disclosure, compared with dose-matched sequential administration of 5-fluorouracil (5-FU) and folinic acid (FA) (‘5-FU:FA’) in animal subjects.

FIG. 2 illustrates pharmacokinetic parameters of 5-FU following bolus or infusion administration of the composition used in the methods of the present disclosure.

FIG. 3 illustrates pharmacokinetic parameters of FUH₂ following bolus or infusion administration of the composition used in the methods of the present disclosure.

FIG. 4 shows in vitro efficacy of the composition used in methods of the present disclosure in tumour models.

FIG. 5 shows in vitro efficacy of the composition used in methods of the present disclosure with a healthy astrocytes derived from a subject.

FIG. 6 shows cell lines at the start and after drug treatment with compositions disclosed herein.

FIG. 7 shows cell lines at the start and after drug treatment with compositions disclosed herein.

FIG. 8 shows an uptake of a composition used in the presently disclosed methods into mouse brain, represented as (A) percent injected dose (% ID) of the composition in the brain or plasma or (B) brain-to-plasma ratio at indicated time-points.

FIG. 9 shows the weight of animals (A) and Deflexifol dose (B) used in a study of mice.

FIG. 10 shows the organ/tissue weights following sacrifice and perfusion. Image (A) shows weight per tissue/organ, and image (B) shows comparison of weights across time point and tissue/organ.

FIG. 11 shows the percent injected dose of Deflexifol as [6-³H]5FU in mouse plasma and brain.

FIG. 12 shows: Percent injected dose of Deflexifol in the kidneys and liver. (A) x-y representation kidneys and (B) column representation of kidneys. (C,E) x-y representation liver and (D, F) column representation liver.

FIG. 13 shows the effect of 5-FU (along or in Deflexifol) on U-87-MG GBM cell proliferation.

FIG. 14 shows IC₅₀ data for Deflexifol and 5-FU.

FIG. 15 shows the status of cells following exposure to various compositions in the presence or absence of 5-FU.

### DETAILED DESCRIPTION

### General Definitions and Terms

With regards to the definitions provided herein, unless stated otherwise, or implicit from context, the defined terms and phrases include the provided meanings. In addition, unless explicitly stated otherwise, or apparent from context, the terms and phrases below do not exclude the meaning that the term or phrase has acquired by a person skilled in the relevant art. The definitions are provided to aid in describing particular embodiments, and are not intended to limit the claimed invention, because the scope of the invention is limited only by the claims.

All publications discussed and/or referenced herein are incorporated herein in their entirety, unless described otherwise.

Unless otherwise required by context, singular terms shall include pluralities and plural terms shall include the singular. Throughout this disclosure, unless specifically stated otherwise or the context requires otherwise, reference to a single step, composition of matter, group of steps or group of compositions of matter shall be taken to encompass one and a plurality (i.e., one or more) of those steps, compositions of matter, groups of steps or groups of compositions of matter. Thus, as used herein, the singular forms “a” “an” and “the” include plural aspects unless the context clearly dictates otherwise. For example, reference to “a” includes a single as well as two or more; reference to “an” includes a single as well as two or more; reference to “the” includes a single as well as two or more and so forth.

Those skilled in the art will appreciate that the disclosure herein is susceptible to variations and modifications other than those specifically described. It is to be understood that the disclosure includes all such variations and modifications. The disclosure also includes all of the examples, steps, features, methods, compositions, formulations, and processes, referred to or indicated in this specification, individually or collectively, and any and all combinations or any two or more of said steps or features.

The term “and/or”, e.g., “X and/or Y” shall be understood to mean either “X and Y” or “X or Y” and shall be taken to provide explicit support for both meanings or for either meaning.

Unless otherwise indicated, the terms “first,” “second,” etc. are used herein merely as labels, and are not intended to impose ordinal, positional, or hierarchical requirements on the items to which these terms refer. Moreover, reference to a “second” item does not require or preclude the existence of lower-numbered item (e.g., a “first” item) and/or a higher-numbered item (e.g., a “third” item).

As used herein, the phrase “at least one of” or “one or more of” when used with a list of items, means different combinations of one or more of the listed items may be used and only one of the items in the list may be needed. The item may be a particular object, thing, or category. In other words, “at least one of” means any combination of items or number of items may be used from the list, but not all of the items in the list may be required. For example, “at least one of item A, item B, and item C” may mean item A; item A and item B; item B; item A, item B, and item C; or item B and item C. In some cases, “at least one of item A, item B, and item C” may mean, for example and without limitation, two of item A, one of item B, and ten of item C; four of item B and seven of item C; or some other suitable combination.

It is to be appreciated that certain features that are, for clarity, described herein in the context of separate embodiments, may also be provided in combination in a single embodiment. Conversely, various features that are, for brevity, described in the context of a single embodiment, may also be provided separately or in any sub-combination.

Throughout the present specification, various aspects and components of the disclosure can be presented in a range format. The range format is included for convenience and should not be interpreted as an inflexible limitation on the scope of the present disclosure. Accordingly, the description of a range should be considered to have specifically disclosed all the possible sub-ranges as well as individual numerical values within that range, unless specifically indicated. For example, description of a range such as from 1 to 5 should be considered to have specifically disclosed sub-ranges such as from 1 to 3, from 1 to 4, from 1 to 5, from 2 to 4, from 2 to 5, from 3 to 5 etc., as well as individual and partial numbers within the recited range, for example, 1, 2, 3, 4, 5, 5.5 and 6, unless where integers are required or implicit from context. This applies regardless of the breadth of the disclosed range. Where specific values are required, these will be indicated in the specification.

Throughout this specification the word “comprise”, or variations such as “comprises” or “comprising”, will be understood to imply the inclusion of a stated element, integer or step, or group of elements, integers or steps, but not the exclusion of any other element, integer or step, or group of elements, integers or steps.

Throughout this specification, the term “consisting essentially of” is intended to exclude elements which would materially affect the properties of the claimed composition, method or process.

The terms “comprising”, “comprise” and “comprises” herein are intended to be optionally substitutable with the terms “consisting essentially of”, “consist essentially of”, “consists essentially of”, “consisting of”, “consist of” and “consists of”, respectively, in every instance.

Herein, unless indicated otherwise, the term “about” encompasses a 10% tolerance in any value or values connected to the term.

Herein “weight %” may be abbreviated to as “wt %” or “wt. %”.

### Chemical and Pharmaceutical Terms

The terms “administration of” and or “administering a” compound, composition or formulation, should be understood to mean providing a compound or composition, as defined herein, to an individual or subject in need thereof.

“Pharmaceutical composition” or “pharmaceutical formulation” refers to a composition suitable for pharmaceutical use in a subject, including humans and mammals. A pharmaceutical composition comprises a pharmacologically effective amount of a composition used in the methods described herein and also comprises a pharmaceutically acceptable carrier. A pharmaceutical composition encompasses a composition comprising the active ingredient(s), and the inert ingredient(s), for example excipients, that make up the carrier, as well as any product which results, directly or indirectly, from combination, complexation or aggregation of any two or more of the ingredients, and/or from dissociation of one or more of the ingredients, and/or from other types of reactions or interactions of one or more of the ingredients. Accordingly, the pharmaceutical compositions of the present invention encompass any composition made by admixing a composition used in the methods described herein and a pharmaceutically acceptable carrier.

“Pharmaceutically acceptable carrier” or merely “carrier”, unless alternatively defined, refers to any of the standard pharmaceutical carriers, buffers, and excipients. The pharmaceutically acceptable carrier may be a saline solution, for example a phosphate buffered saline solution, or a 5% aqueous solution of dextrose. Further examples of a pharmaceutically acceptable carrier include but are not limited to emulsions, such as an oil/water or water/oil emulsion, and various types of wetting agents and/or adjuvants. Suitable pharmaceutical carriers and formulations are described in Remington's Pharmaceutical Sciences, 19^{th} Ed. (Mack Publishing Co., Easton, 1995). Preferred pharmaceutical carriers depend upon the intended mode of administration of the active agent. Example modes of administration include enteral (e.g., oral) or parenteral (e.g., subcutaneous, intramuscular, intravenous or intraperitoneal injection; or topical, transdermal, or transmucosal administration).

As used herein, “pharmaceutically acceptable salt” refers a salt that can be formulated into a compound for pharmaceutical use including, e.g., metal salts (sodium, potassium, magnesium, calcium, etc.) and salts of ammonia or organic amines.

As used herein the term “complex” is understood to mean a non-covalent physical interaction between two or more chemical entities.

As used herein, the term “Fluorodex” (abbreviation FD) or “Deflexifol” is understood to mean a composition containing 5-FU and folinic acid salt together with a cyclodextrin or derivative thereof in aqueous solution, as disclosed herein.

As used herein, the term “synergistic” refers to a greater than additive effect that is produced by a combination of two or more compounds (for example folinic acid, or a salt thereof, and 5-FU, or a salt thereof), which exceeds the effect that would otherwise result from use of the two or more compounds (for example folinic acid or 5-FU), alone.

A “therapeutically effective amount”, or “effective amount” as used herein, includes within its meaning a non-toxic but sufficient amount of:
(a) a composition comprising: 5-fluorouracil (5-FU), a pharmaceutically acceptable salt thereof, or a pharmaceutically acceptable analogue thereof; or folinic acid, or a pharmaceutically acceptable salt thereof;
(b) 5-fluorouracil (5-FU), a pharmaceutically acceptable salt thereof, or a pharmaceutically acceptable analogue thereof; and/or
(c) folinic acid, or a pharmaceutically acceptable salt thereof,
   to provide the desired therapeutic or prophylactic effect. The phrases are taken to mean an amount of, for example, 5-FU and folinic acid that will elicit a desired biological or medical response of a tissue, system, animal or human that is being sought by the researcher, veterinarian, medical doctor or other clinician administering the compound of a composition comprising the compound. The exact amount required will vary from subject to subject depending on factors such as the subject's general health, the subject's age, the subject's gender, the subject's body surface area, the stage and severity of the cancer, as well as any contraindications.

The recipients of the recited compounds and pharmaceutically acceptable compositions are referred herein with the interchangeable terms “patient”, “recipient” “individual”, and “subject”. These four terms are used interchangeably and refer to any human or animal (unless indicated otherwise), as defined herein.

The recipients of a composition or pharmaceutical composition described herein, can be a human being, male or female. The recipient may be a child less than 18 years old. For example the recipient may be less than 16, 14, 12, 10, 8 or 6 years old. The recipient may be an adult, for example an recipient that is 18 years old or older.

In one embodiment, the subject or recipient of a composition described herein is free of one or more body cancers, for example a cancer of the colon, breast, head, neck and/or pancreas. In another, the subject or recipient of a composition described herein is free of one or more cancers selected from: breast cancers; digestive/gastrointestinal cancers; endocrine and/or neuroendocrine cancers; eye cancers; genitourinary cancers; germ cell cancers; gynaecological cancers; head and/or neck cancers; hematologic/blood cancers; musculoskeletal cancers; respiratory/thoracic cancers; and/or skin cancers.

Alternatively the recipients of a composition or pharmaceutical composition described herein, e.g., a patient or subject, can also be a non-human animal. “Non-human animals” or “non-human animal” is directed to the kingdom Animalia, excluding humans, and includes both vertebrates and invertebrates, male or female, and comprises: warm blooded animals, including mammals (comprising but not limited to primates, dogs, cats, cattle, pigs, sheep, goats, rats, guinea pigs, horses, or other bovine, ovine, equine, canine, feline, rodent or murine species), birds, insects, reptiles, fish and amphibians.

When the recipient is a non-human animal, possible advantages can include one or more of the ability to administer a composition via bolus; less potential side effects due to physiological pH; and/or by providing an all in one solution versus separate administration of, for example, 5-FU and LV, to an animal.

As used herein, the term “prevention” includes either preventing the onset of a clinically evident cancer altogether, or delaying its onset. For prophylactic benefit, the composition used in the methods described herein may be administered to a patient at risk of developing a brain and/or CNS cancer, or to a patient reporting one or more of the physiological symptoms of such a disease, even though a diagnosis of the disease may not have been made.

As used herein, the term “treatment” includes partial or total inhibition of cancer growth and spread, such as local invasion and distant metastasis, as well as partial or total destruction of the cancer cells. Treatment as used herein includes prophylactic treatment as well as therapeutic treatment. A “prophylactic” treatment is a treatment administered to a subject who does not exhibit signs of a disease or exhibits only early signs for the purpose of decreasing the risk of developing pathology. A composition used in the methods described herein may be given as a prophylactic treatment to reduce the likelihood of developing a pathology or to minimize the severity of the pathology, if developed. A “therapeutic” treatment is a treatment administered to a subject who exhibits signs or symptoms of pathology for the purpose of diminishing or eliminating those signs or symptoms. The signs or symptoms may be biochemical, cellular, histological, functional, subjective or objective. The composition used in the methods described herein may be given as a therapeutic treatment.

### Compositions

Disclosed herein are methods and uses of compositions, the compositions comprising:
(i) 5-FU, a pharmaceutically acceptable salt thereof, or a pharmaceutically acceptable analogue thereof;
(ii) folinic acid, or a pharmaceutically acceptable salt thereof; and
(iii) a cyclodextrin, or a pharmaceutically acceptable salt or derivative thereof, for the treatment of cancer of the brain or central nervous system (CNS).

The compositions were first disclosed in WO 2008/106721, the content of which is incorporated herein in its entirety by reference.

In one embodiment, the combination of said ingredients prevents 5-FU from precipitating out of solution at a pH of about 5 to about 9.

In another embodiment the compositions described herein are homogenous.

In the present specification, reference to 5-FU will be taken to include a corresponding reference to a pharmaceutically acceptable salt thereof or an analogue thereof, reference to folinic acid will be taken to include a corresponding reference to a pharmaceutically acceptable salt or derivative thereof, and reference to cyclodextrin will be taken to include a corresponding reference to a pharmaceutically acceptable salt or derivative thereof, unless the contrary is indicated or implied by context.

### 5-Fluorouracil

5-Fluorouracil (5-FU) is a nucleoside metabolic inhibitor. 5-FU is available, for example, as a colourless to faint yellow, aqueous, sterile, non-pyrogenic injectable solution available in a pharmacy bulk package, a sterile preparation that contains doses for multiple patients for intravenous administration. In one embodiment, each mL contains 50 mg 5-FU in water for injection, USP. The pH can be adjusted to approximately 9.2 with sodium hydroxide. Chemically, 5-FU is 5-fluoro-2,4 (1H,3H)-pyrimidinedione.

5-FU is a fluorinated pyrimidine and belongs to a class of chemotherapy compounds termed fluoropyrimidines.

Analogues of 5-FU are suitable for use in the methods disclosed herein. In the present context, an analogue of 5-FU is a compound having comparable or similar pharmacological activity, particularly in a human patient, to 5-FU, and similar solubility/complexation properties, so that in a composition according to the present disclosure, when made with the analogue, the analogue remains in solution. The analogue of 5-FU may be, for example, 5-fluoro-2′-deoxyuridine (5-FUdr).

The 5-FU present in the compositions of the present disclosure may be present as a pharmaceutically acceptable salt. By “pharmaceutically acceptable salt” it is meant those salts of 5-FU which are suitable for use in contact with the tissues of humans and other animals without undue toxicity, irritation, and/or allergic response, and/or the like, and are commensurate with a reasonable benefit/risk ratio. Pharmaceutically acceptable salts are well known in the art.

For instance, suitable pharmaceutically acceptable salts of 5-FU include the sodium salt, which may be obtained from reaction with sodium hydroxide.

### Folinic Acid

The compositions herein comprise folinic acid, or a pharmaceutically acceptable salt thereof. Folinic acid may be administered as a salt, for example a pharmaceutically acceptable salt. The folinic acid present in the composition may be present as a salt of an alkali metal (e.g., sodium or potassium) or a salt of an alkaline earth metal (e.g., magnesium or calcium). In one embodiment, the folinic acid may be present as calcium folinate (leucovorin calcium) or sodium folinate (leucovorin sodium). The salt may be in the form of a hydrate, for example calcium folinate pentahydrate. Herein, leucovorin may be potentially referred to as “LV”.

Herein, the folinic acid, or pharmaceutically acceptable salt thereof, may be present in either of its enantiomeric forms, or as a racemic mixture. It may be the 6(S) diastereomer or the 6(R) diastereomer (wherein the glutamic acid residue of the folinic acid or folinate is in the L form), or it may be a mixture of these in any desired ratio, such as about 1:1 or some other ratio. It may be a mixture of these in which the 6(S) isomer is enriched. It may be between about 51 and about 100% enriched, or between about 55 and 99%, 55 and 95%, 55 and 75%, 55 and 65%, 75 and 85%, 75 and 95%, 85 and 95% or 75 and 99% enriched, e.g., about 51, 52, 53, 54, 55, 60, 65, 70, 75, 80, 85, 90, 95, 96, 97, 98, 99 or 100% enriched. It should be noted that the 6(S) form having the glutamic acid residue in the L form is pharmaceutically active.

### Cyclodextrins

In the compositions of the present disclosure, cyclodextrin (optionally abbreviated to “CD” herein), and 5-FU may form a complex in solution. This complex may be a “host-guest” or “inclusion complex”. The complex may result in the otherwise insoluble 5-FU being soluble in aqueous solution at pH values of between about 5 to about 8. Alternatively, the cyclodextrin and 5-FU may be associated through some other form of non-covalent interaction. Cyclodextrin and folinic acid may also form a complex in solution. This complex may be a “host-guest” or “inclusion complex”. The 5-FU and cyclodextrin may be present in the composition in the form of a non-covalent complex, i.e., the 5-FU may be complexed to the cyclodextrin but not covalently bound thereto.

Cyclodextrins suitable for use in accordance with the present disclosure include natural and chemically modified cyclodextrins. The cyclodextrin may be: an α-cyclodextrin, a β-cyclodextrin, a γ-cyclodextrin, or a derivative or pharmaceutically acceptable salt thereof. Mixtures of any two or more of the above cyclodextrins are also contemplated in the compositions of the present disclosure.

Derivatives of cyclodextrins that may be used in the compositions may include cyclodextrins whereby one, or some, or all of the hydroxy groups are converted to other functional groups. As a result, the degree of substitution of the cyclodextrin may vary.

In one embodiment, one or more of the hydroxy groups may be converted to OR groups. R may be a hydrocarbon group having between 1 and 20 carbon atoms, or between 1 and 15, or between 1 and 10, or between 1 and 9, or between 1 and 8, or between 1 and 7, or between 1 and 6, or between 1 and 5, or between 1 and 4 carbon atoms, for example methyl, allyl, ethyl, propyl, isopropyl, propargyl, butyl, but-2-enyl, secbutyl, pentyl, 3-methyl-pent-2-enyl, hexyl, heptyl, octyl, nonyl, decyl etc. In one embodiment, the hydrocarbon group is a branched or straight chain alkyl group. The hydrocarbon group may be optionally substituted with one or more halo groups and/or one or more hydroxy groups, such that R is a halo-substituted hydrocarbon or a hydroxy-substituted hydrocarbon, for example a hydroxyalkyl group. Examples of hydroxyalkyl groups include, but are not limited to —CH₂CH(OH)CH₃, —CH₂CH₂OH, —CH₂CH₂CH₂(OH)CH₃, —CH(OH)CH₂CH₂CH₃ and —CH(OH)CH₂CH₂CH₂CH₃. In one embodiment, the cyclodextrin is a hydroxyalkyl β-cyclodextrin, for example hydroxypropyl-β-cyclodextrin, such as 2-hydroxypropyl-β-cyclodextrin.

Derivatives of cyclodextrins also include anionic cyclodextrins such as carboxymethyl cyclodextrins, cyclodextrin sulfates, cyclodextrin sulfonates, cyclodextrin phenolates and cyclodextrin phosphates, or mixtures thereof. In one embodiment, the cyclodextrin derivative may be a sulfated α, β-, or γ-cyclodextrin, or a salt thereof, for example a sodium salt. The sulfated cyclodextrin may be polysulfated or comprise a mixture of sulfate and hydroxy groups. In one embodiment, the sulfated cyclodextrin is a β-cyclodextrin comprising between 3 and 20, or between 3 and 18, or between 4 and 16, or between 4 and 14, or between 5 and 13, or between 5 and 12, or between 5 and 11, or between 6 and 10, or between 7 and 9, or between 9 and 12, or between 5 and 10 sulfate groups. The sulfate groups may be present as salts, for example sodium salts. In one embodiment, the cyclodextrin derivative is heptakis(6-O-sulfo)-β-cyclodextrin, or a salt thereof.

The cyclodextrin may be a cyclodextrin sulfate, or a salt thereof. The β-cyclodextrin sulfate salt may be a sodium salt. In another embodiment the β-cyclodextrin sulfate salt may be a heptakis(6-O-sulfo)-β-cyclodextrin, for example the sodium salt.

Further cyclodextrin derivatives which may be used include sulfoether cyclodextrins such as sulfoalkylether cyclodextrins, wherein the alkyl group comprises between 1 and 15 carbon atoms, or between 1 and 12, or between 1 and 10, or between 1 and 9, or between 1 and 8, or between 1 and 7, or between 1 and 6, or between 1 and 5, or between 1 and 4 carbon atoms, for example sulfobutyl β-cyclodextrin (Captisol®). The alkyl group may be of the formula —(CH₂)ₙ—, wherein n is a number between 1 and 8, or between 1 and 7, or between 1 and 6, or between 1 and 5 or between 1 and 4.

Additional cyclodextrin derivatives that may be suitable for use in the compositions of the present disclosure are those disclosed in “Chemical Reviews: Cyclodextrins”, V. T. D'Souza and K. B. Lipkowitz, Vol. 98, No. 5 (American Chemical Society, 1998), which is incorporated herein by reference.

As noted above, the degree of substitution of the cyclodextrin may vary. Suitable cyclodextrins therefore include for example those cyclodextrins wherein one, or some, or all of the hydroxy groups are substituted. The degree of substitution may be between about 1 and 20, or between about 5 and 20, 10 and 20, 1 and 10, 1 and 5, 5 and 15, 5 and 10 or 10 and 15, e.g., about 1, 2, 3, 4, 5, 6, 7, 8, 9, 10, 11, 12, 13, 14, 15, 16, 17, 18, 19 or 20. The cyclodextrin may be a mixture of cyclodextrins of different degrees of substitution. In such a case the average degree of substitution may be between about 1 and 20, or between about 5 and 20, 10 and 20, 1 and 10, 1 and 5, 5 and 15, 5 and 10 or 10 5 and 15, e.g., about 1, 2, 3, 4, 5, 6, 7, 8, 9, 10, 11, 12, 13, 14, 15, 16, 17, 18, 19 or 20. In one embodiment where the cyclodextrin is a hydroxypropyl-β-cyclodextrin, the average degree of substitution may be between about 4 and about 12, or between about 5 and about 10, or between about 5 and about 9, or between about 6 and about 8, or about 7.

In one embodiment, the amount of cyclodextrin used in the compositions of the present disclosure is an amount sufficient to ensure that the 5-FU present in the compositions is maintained in solution. The molar ratio of CD:5-FU may be between about 1:10 and about 3:1, or between about 1:10 and 1:1, 1:10 and 1:2, 1:10 and 1:5, 1:5 and 3:1, 1:5 and 1:1, 1:3 and 3:1, 1:8 and 3:1, 1:8 and 2:1, 1:8 and 1:1, 1:8 and 1:2, 1:8 and 1:5, 1:3 and 2:1, 1:3 and 1:1, 1:3 and 1.1:1, 1:3 and 1.2:1, 1:3 and 1.3:1, 1:3 and 1.4:1, 1:3 and 1.5:1, 1:3 and 2:1.5, 1:3 and 2.5:1, 1:2.5 and 3:1, 1:2 and 3:1, 1:1.5 and 3:1, 1:1.4 and 3:1, 1:1.3 and 3:1, 1:1.2 and 3:1, 1:1.1 and 3:1, 1:1 and 3:1, 1.5:1 and 3:1, 2:1 and 3:1, 2.5:1 and 3:1, 2:1 and 1:2, 1.5:1 and 1:1.5, 1.4:1 and 1:1.4, 1.3:1 and 1:1.3, 1.2:1 and 1:1.2 or 1.1:1 and 1:1.1, e.g., about 1:10, 1:9, 1:8, 1:7, 1:6, 1:5, 1:4.5, 1:4, 1:3.5, 1:3, 1:2.5, 1:2, 1:1.5, 1:1.4, 1:1.3, 1:1.2, 1:1.1, 1:1, 1.1:1, 1.2:1, 1.3:1, 1.4:1, 1.5:1, 2:1, 2.5:1 or 3:1. This ratio may depend on the particular CD used.

In one embodiment, the cyclodextrin is a sulfated sodium salt of β-cyclodextrin, optionally a polysulfated sodium salt or a mixture of sulfated sodium salts with differing degrees of sulfation. In another embodiment, the cyclodextrin is a hydroxyalkyl O-cyclodextrin, for example hydroxypropyl-β-cyclodextrin. Alternatively, the cyclodextrin may be a mixture of different types of cyclodextrins, for example a mixture of hydroxyalkyl β-cyclodextrin and sulfated β-cyclodextrin.

pH

The pH of a composition disclosed herein may be about 5.0 to about 9.0, or about 6.5 to about 8, or about 7.0 to about 7.8.

A composition disclosed herein may be mildly or moderately acidic, neutral or mildly or moderately basic. The pH of the composition may be: between about 5.0 and about 9.0, or between about 5.0 and about 8.0, or between about 5.0 and about 7.0, or between about 5.5 and about 8.0, or between about 6.0 and about 8.0, or between about 6.5 and about 5 8.0, or between about 7.0 and about 8.0, or between 7.5 and about 8.0, or between about 7.0 and about 9.0, or between about 7.5 and 7.8, or between about 7.5 and about 7.7, or between about 7.6 and about 7.7, or between about 6.7 and about 7.7, or between about 6.8 and about 7.6, or between about 7.0 and. about 7.6, or between about 7.2 and about 7.6, or between about 7.3 and about 7.6, or between about 7.3 and 7.5. For example, the pH may be about, or at least about: 5.0, 5.1, 5.3, 5.5, 5.7, 5.9, 6.1, 6.3, 6.5, 6.7, 6.9, 7.0, 7.1, 7.2, 7.3, 7.4, 7.5, 7.6, 7.7, 7.8, 7.9, 8.0, 8.1, 8.2, 8.3, 8.4, 8.5, 8.6, 8.7, 8.8, 8.9 or 9.0.

### Pharmaceutical Compositions

Disclosed herein are methods relating to the treatment and/or prevention of a cancer of the brain or CNS in a patient, for example by administering to the patient a therapeutically effective amount of a pharmaceutical composition comprising:
(i) 5-FU, a pharmaceutically acceptable salt thereof, or a pharmaceutically acceptable analogue thereof;
(ii) folinic acid, or a pharmaceutically acceptable salt thereof; and
(iii) a cyclodextrin, or a pharmaceutically acceptable salt or derivative thereof.

Also disclosed herein is use of a composition comprising:
(i) 5-FU, a pharmaceutically acceptable salt thereof, or a pharmaceutically acceptable analogue thereof;
(ii) folinic acid, or a pharmaceutically acceptable salt thereof; and
(iii) a cyclodextrin or a pharmaceutically acceptable salt thereof.
   in the manufacture of a medicament for the treatment and/or prevention of a cancer of the brain and/or central nervous system.

In some embodiments, compositions can be administered to a patient already suffering from cancer, in an amount sufficient to cure, or at least partially arrest the cancer and its complications. The compositions should provide a quantity of (i) 5-FU, a pharmaceutically acceptable salt thereof, or a pharmaceutically acceptable analogue thereof; and (ii) folinic acid, or a pharmaceutically acceptable salt thereof, sufficient to effectively treat, or partially treat, the cancer.

In some embodiments, the methods disclosed herein also include prophylactic or preventative application of the compositions. In some embodiments, the compositions are used as a maintenance therapy.

In general, suitable compositions for use in accordance with the methods of the present disclosure may be prepared according to methods and procedures that are known to those of ordinary skill in the art, and accordingly may include a pharmaceutically acceptable carrier, diluent and/or adjuvant. The diluents, adjuvants and excipients must be “acceptable” in terms of being compatible with the other components of the composition, and not deleterious to the recipient thereof.

Compositions may be administered by standard routes or means. In general, the compositions may be administered intravenously, intraperitoneally, intraarterially, or topically. They may also be administered intramuscularly, subcutaneously or transdermally (e.g., by means of a patch). Administration may, for example, be systemic or regional. The particular route of administration to be used in any given circumstance will depend on a number of factors, including, but not limited to: the type of cancer being treated; the severity and extent of the cancer; the required dosage of, for example 5-FU and folinic acid, which need to be delivered; and any potential side-effects.

The composition may comprise one or more pharmaceutically acceptable carrier, diluent and/or adjuvant. The carrier may be an aqueous carrier, e.g., saline solution. The composition may be an aqueous composition.

Examples of pharmaceutically acceptable carriers or diluents are demineralised or distilled water, BP or USP water, saline solution, Ringer's solution, glucose solution, vegetable based oils such as peanut oil, safflower oil, olive oil, cottonseed oil, maize oil, sesame oils such as peanut oil, safflower oil, olive oil, cottonseed oil, maize oil, sesame oil, arachis oil or coconut oil; silicone oils, including polysiloxanes, such as methyl polysiloxane, phenyl polysiloxane and methylphenyl polysolpoxane; volatile silicones; mineral oils such as liquid paraffin, soft paraffin or squalane; cellulose derivatives such as methyl cellulose, ethyl cellulose, carboxymethylcellulose, sodium carboxymethylcellulose or hydroxypropylmethylcellulose; lower alkanols, for example ethanol or iso-propanol; lower aralkanols; lower polyalkylene glycols or lower alkylene glycols, for example polyethylene glycol, polypropylene glycol, ethylene glycol, propylene glycol, 1,3-butylene glycol or glycerin; fatty acid esters such as isopropyl palmitate, isopropyl myristate or ethyl oleate; polyvinylpyrrolidone; agar; carrageenan; gum tragacanth or gum acacia, and petroleum jelly. The carrier or carriers may form from about 10% to about 99.9% by weight of the compositions.

The compositions of the present disclosure may be in a form suitable for administration by injection, in the form of an ointment, cream or lotion suitable for topical administration, or in a form suitable for administration by injection intravenously, intraperitoneally or intraarterially.

For administration as an injectable solution or suspension, non-toxic parenterally acceptable diluents or carriers can comprise: Ringer's solution, isotonic saline, glucose solution, distilled water, phosphate buffered saline, ethanol and 1,2-propylene glycol.

Adjuvants may comprise: emollients, emulsifiers, thickening agents, preservatives, bactericides and/or buffering agents.

Topical formulations comprise the composition of the present disclosure together with one or more acceptable carriers, and optionally any other therapeutic ingredients. Formulations suitable for topical administration include liquid or semi-liquid preparations suitable for penetration through the skin to the site of where treatment is required, such as liniments, lotions, creams, ointments or pastes.

Lotions according to the present disclosure include those suitable for application to the skin. Lotions or liniments for application to the skin may also include an agent to hasten drying and to cool the skin, such as an alcohol or acetone, and/or a moisturizer such as glycerol, or oil such as castor oil or arachis oil.

The compositions may incorporate any suitable surfactant such as an anionic, cationic or non-ionic surfactant such as sorbitan esters or polyoxyethylene derivatives thereof. Suspending agents such as natural gums, cellulose derivatives or inorganic materials such as silicaceous silicas, and other ingredients such as lanolin, may also be included.

The composition may be in the form of a solution, e.g., an aqueous solution. For example, one, two or each of the components:
(i) 5-FU, a pharmaceutically acceptable salt thereof, or a pharmaceutically acceptable analogue thereof;
(ii) folinic acid, or a pharmaceutically acceptable salt thereof; and
(iii) a cyclodextrin or a pharmaceutically acceptable salt thereof;
   may be in solution in the composition.

In some embodiments, the composition is homogeneous. In some embodiments, the composition may contain no solid matter. Thus each of the components (i), (ii), and (iii) above, may be completely in solution in the composition.

The composition may be a synergistic composition.

Examples of possible compositions are provided herein. Unless dictated by context or by specific directions, these exemplified compositions may be utilised in the methods and/or treatments described herein, or used in the formation of a medicament for the methods and/or treatments described herein.

In another embodiment there is provided a composition comprising, or consisting of
(i) 5-FU or a pharmaceutically acceptable salt or analogue thereof;
(ii) folinic acid, or a pharmaceutically acceptable salt thereof;
(iii) a cyclodextrin or a pharmaceutically acceptable salt or derivative thereof; and
(iv) a pharmaceutically acceptable carrier;
   wherein (i), (ii), and (iii) are dissolved in (iv). In addition, the cyclodextrin may be selected from the group consisting of a hydroxyalkyl β-cyclodextrin and a sulfated β-cyclodextrin, or a combination thereof. The hydroxyalkyl β-cyclodextrin may be hydroxypropyl-β-cyclodextrin, or more specifically 2-hydroxypropyl-β-cyclodextrin.

For the preparation of the compositions disclosed herein, it should be noted that, unless dictated specifically or by context, when preparing the composition, components (i), (ii), (iii) and (iv) may be mixed in any order. In addition, unless dictated specifically or by context, further components may be added or introduced before, during or after any step comprising one or more of components (i), (ii), (iii) and (iv).

In another embodiment there is provided a composition comprising, or consisting of:
(i) 5-FU or a pharmaceutically acceptable salt or analogue thereof in a concentration of between about 1 and about 50 mg/ml;
(ii) folinic acid, or a pharmaceutically acceptable salt thereof, in a concentration of between about 0.1 and about 5 mg/ml;
(iii) a cyclodextrin or a pharmaceutically acceptable salt or derivative thereof in a concentration of between about 10 and about 300 mg/ml; and
(iv) a pharmaceutically acceptable carrier;
   wherein (i), (ii), and (iii) are dissolved in (iv).

In another embodiment there is provided a composition comprising, or consisting of:
(i) 5-FU or a pharmaceutically acceptable salt or analogue thereof in a concentration of between about 10 and about 20 mg/ml;
(ii) folinic acid, or a pharmaceutically acceptable salt thereof, in a concentration of between about 0.5 and about 2 mg/ml;
(iii) a cyclodextrin or a pharmaceutically acceptable salt or derivative thereof in a concentration of between about 20 and about 200 mg/ml; and
(iv) a pharmaceutically acceptable carrier,
   wherein (i), (ii), and (iii) are dissolved in (iv). The cyclodextrin may be selected from the group consisting of: a hydroxyalkyl β-cyclodextrin and a sulfated β-cyclodextrin, or a combination thereof. The hydroxyalkyl β-cyclodextrin may be hydroxypropyl-β-cyclodextrin.

In another embodiment there is provided a composition comprising, or consisting of:
(i) 5-FU at a concentration of about 15 mg/ml;
(ii) folinic acid calcium salt at a concentration of about 1 mg/ml;
(iii) β-cyclodextrin sulfate salt at a concentration of about 45 mg/ml; and
(iv) a pharmaceutically acceptable carrier,
   wherein (i), (ii), and (iii) are dissolved in (iv).

In another embodiment there is provided a composition comprising, or consisting of:
(i) 5-FU at a concentration of about 15 mg/ml;
(ii) folinic acid calcium salt at a concentration of about 1 mg/ml;
(iii) β-cyclodextrin sulfate salt at a concentration of about 175 mg/ml; and (iv) a pharmaceutically acceptable carrier,
   wherein (i), (ii), and (iii) are dissolved in (iv).

In another embodiment there is provided a composition comprising, or consisting of:
(i) 5-FU at a concentration of about 45 mg/ml;
(ii) folinic acid calcium salt at a concentration of about 1 mg/ml;
(iii) β-cyclodextrin sulfate salt at a concentration of about 45 mg/ml; and
(iv) a pharmaceutically acceptable carrier,
   wherein (i), (ii), and (iii) are dissolved in (iv). The β-cyclodextrin sulfate salt may comprise between 6 and 14 sulfate groups. The pharmaceutically acceptable carrier may be 0.9% saline solution.

In another embodiment there is provided a composition comprising, or consisting of:
(i) 5-FU at a concentration of about 15 mg/ml;
(ii) folinic acid calcium salt at a concentration of about 1 mg/ml;
(iii) 2-hydroxypropyl-β-cyclodextrin at a concentration of about 100 mg/mL; and
(iv) a pharmaceutically acceptable carrier,
   wherein (i), (ii) and (iii) are dissolved in (iv). The pharmaceutically acceptable carrier may be 0.9% saline solution.

In another embodiment there is provided a composition comprising, or consisting of:
(i) 5-FU at a concentration of about 15 mg/ml;
(ii) folinic acid calcium salt at a concentration of about 1 mg/ml;
(iii) heptakis(6-O-sulfo)-β-cyclodextrin salt at a concentration of about 50 mg/mL; and
(iv) a pharmaceutically acceptable carrier,
   wherein (i), (ii) and (iii) are dissolved in (iv). The pharmaceutically acceptable carrier may be 0.9% saline solution. The heptakis(6-O-sulfo)-β-cyclodextrin salt may be a sodium salt.

### Dosage

In some embodiments, compositions can be administered to a patient already suffering from cancer, in an amount sufficient to cure, or at least partially arrest the cancer and its complications. The composition should provide a quantity of: (i) 5-FU, a pharmaceutically acceptable salt thereof, or a pharmaceutically acceptable analogue thereof; and (ii) folinic acid, or a pharmaceutically acceptable salt thereof, sufficient to effectively treat the cancer.

In some embodiments, the methods, compositions and uses disclosed herein also include or relate to prophylactic or preventative application of a suitable effective dose of the composition. In some embodiments, the composition, in an appropriate effective dose, is used as a maintenance therapy.

The therapeutically effective dose level for any particular patient or subject, will depend upon a variety of factors familiar to one skilled in the art, including, for example: the cancer being treated and/or the severity of the cancer, the age, body weight, general health, sex and diet of the patient, the time of administration, the route of administration, the duration of the treatment, drugs used in combination or coincidental with the treatment, together with other related factors well known in medicine. It will be understood, however, that the specific dose level and frequency of dosage for any particular patient may be varied and will depend upon a variety of factors including the activity of the specific compound employed, the metabolic stability and length of action of that compound, the age, body weight, general health, gender, diet, mode and time of administration, rate of excretion, drug combination, the severity of the particular condition, a patient's body surface area, whether the patient is undergoing therapy, and any specific contraindications.

In consideration of the factors described above, one skilled in the art would be able, by routine experimentation in accordance with their common general knowledge, to determine an effective, non-toxic amount of the composition which would be required to prevent and/or treat one or more of the disclosed diseases and conditions. For example, in a therapeutic application, a patient may be administered a maximum safe dose based on their body surface area (for example intravenously). In some embodiments, a maximum safe dose may be administered intravenously, for example as a bolus dose, which may be given by infusion or injection, followed by an IV infusion.

In some embodiments, the composition is administered either as an intravenous (IV) bolus and/or as an IV infusion. In some embodiments, the composition may be administered via a bolus IV, bolus IV followed by IV infusion, or IV infusion regimen. For example, in some embodiments, the composition may be administered as a bolus IV or a bolus IV followed by IV infusion, for example over the period of about 24 hours to about 48 hours. Alternatively, in some embodiments, the composition may be administered as an IV infusion only. IV infusion regimes may vary in the timeframe, which for example includes any period within about 24 hours to about 48 hours.

In some embodiments a composition as defined herein may be administered (e.g., as a bolus IV and/or an IV infusion) at a dose of from about 400 mg/m² to about 3000 mg/m² or about 3500 mg/m², for example: 450, 475, 500, or 525 00 mg/m² to about 3000 mg/m² or about 3500 mg/m². For example at a dose of about, or at least about: 400 mg/m²; 425 mg/m²; 450 mg/m²; 475 mg/m²; 500 mg/m²; 525 mg/m²; 550 mg/m²; 575 mg/m²; 600 mg/m²; 700 mg/m²; 800 mg/m²; 900 mg/m²; 1000 mg/m²; 1100 mg/m²; 1200 mg/m²; 1300 mg/m²; 1400 mg/m²; 1500 mg/m²; 1600 mg/m²; 1700 mg/m²; 1800 mg/m²; 1900 mg/m²; 2000 mg/m²; 2100 mg/m²; 2200 mg/m²; 2300 mg/m²; 2400 mg/m²; 2500 mg/m²; 2600 mg/m²; 2700 mg/m²; 2800 mg/m²; 2900 mg/m²; 3000 mg/m²; 3100 mg/m²; 3200 mg/m²; 3300 mg/m²; 3400 mg/m²; 3500 mg/m², 3600 mg/m², 3700 mg/m², or 3800 mg/m², or any ranges using two of these values.

In another embodiment, an effective dosage of 5-FU, a pharmaceutically acceptable salt thereof, or a pharmaceutically acceptable analogue thereof, may be in the range of about 0.0001 mg to about 1000 mg per kg body weight per 24 hours; for example, about 0.001 mg to about 750 mg per kg body weight per 24 hours; about 0.01 mg to about 500 mg per kg body weight per 24 hours; about 0.1 mg to about 500 mg per kg body weight per 24 hours; about 0.1 mg to about 250 mg per kg body weight per 24 hours; about 1.0 mg to about 250 mg per kg body weight per 24 hours. Alternatively, an effective dose range may be in the range about 1.0 mg to about 200 mg per kg body weight per 24 hours; about 1.0 mg to about 100 mg per kg body weight per 24 hours; about 1.0 mg to about 50 mg per kg body weight per 24 hours; about 1.0 mg to about 25 mg per kg body weight per 24 hours; about 5.0 mg to about 50 mg per kg body weight per 24 hours; about 5.0 mg to about 20 mg per kg body weight per 24 hours; about 5.0 mg to about 15 mg per kg body weight per 24 hours; or about 5.0 to 10.0 mg/kg body weight per 24 hours.

In yet another embodiment, an effective dosage of folinic acid, or a pharmaceutically acceptable salt thereof, may be in the range of about 0.0001 mg to about 1000 mg per kg body weight per 24 hours; for example, about 0.001 mg to about 750 mg per kg body weight per 24 hours; about 0.01 mg to about 500 mg per kg body weight per 24 hours; about 0.1 mg to about 500 mg per kg body weight per 24 hours; about 0.1 mg to about 250 mg per kg body weight per 24 hours; about 1.0 mg to about 250 mg per kg body weight per 24 hours. Alternatively, an effective dose range may be in the range about 1.0 mg to about 200 mg per kg body weight per 24 hours; about 1.0 mg to about 100 mg per kg. body weight per 24 hours; about 1.0 mg to about 50 mg per kg body weight per 24 hours; about 1.0 mg to about 25 mg per kg body weight per 24 hours; about 5.0 mg to about 50 mg per kg body weight per 24 hours; about 5.0 mg to about 20 mg per kg body weight per 24 hours; about 5.0 mg to about 15 mg per kg body weight per 24 hours. It may be a low dose or high dose folinic acid composition.

In a composition according to the present disclosure the: 5-FU, a pharmaceutically acceptable salt thereof, or a pharmaceutically acceptable analogue thereof; and the folinic acid, or a pharmaceutically acceptable salt thereof, may be present in pharmaceutically effective concentrations. The 5-FU, a pharmaceutically acceptable salt thereof, or a pharmaceutically acceptable analogue thereof, may be present in a concentration of between about 1 and about 50 mg/ml, or between about 1 and 40, 1 and 30, 1 and 20, 1 and 10, 1 and 5, 5 and 50, 10 and 50, 20 and 50, 5 and 30, 5 and 20 or 10 and 20 mg/ml, e.g., about, or at least about: 1, 2, 3, 4, 5, 6, 7, 8, 9, 10, 11, 12, 13, 14, 15, 16, 17, 18, 19, 20, 25, 30, 35, 40, 45 or 50 mg/ml. The folinic acid or a pharmaceutically acceptable salt thereof may be present at a concentration of between about 0.1 and about 25 mg/ml or between about 0.1 and about 15 mg/ml or between about 0.1 and about 12 mg/ml. Alternatively, the folinic acid or a pharmaceutically acceptable salt thereof, may be present in a concentration of between about 0.01 and about 25 mg/ml, or between about 0.01 and 25, 0.1 and 20, 0.1 and 15, 0.1 and 10, 0.1 and 5, 0.1 and 2, 0.1 and 1, 1 and 25, 5 and 25, 10 and 25, 15 and 25, 1 and 15, 1 and 10, 1 and 5, 0.5 and 5, 0.5 and 2, 5 and 20, 5 and 10 or 10 and 20 mg/ml, e.g., about, or at least about: 0.1, 0.2, 0.3, 0.4, 0.5, 0.6, 0.7, 0.8, 0.9, 1, 2, 3, 4, 5, 6, 7, 8, 9, 10, 15, 20 or 25 mg/ml. In one embodiment, the 5-FU is present in a concentration of about 15 mg/ml and the folinic acid is present in an amount of about 1 mg/ml.

In another embodiment, the ratio of the concentrations of the: 5-FU, a pharmaceutically acceptable salt thereof, or a pharmaceutically acceptable analogue thereof; and the folinic acid, or a pharmaceutically acceptable salt thereof, is about, or at least about: 20:1, 19:1, 18:1, 17:1, 16:1, 15:1, 14:1, 13:1, 12:1, 11:1, 10:1, 9:1, 8:1, 7:1, 6:1, 5:1, 4:1, 3:1, 2:1, or 1:1. In one embodiment the ratio of the concentrations of the 5-FU to folinic acid is about, or at least about, 15:1.

Herein, the CD, or a pharmaceutically acceptable salt thereof, may be present in a concentration of between about 1 and about 300 mg/ml, or between about 1 and 250, 1 and 200, 1 and 150, 1 and 100, 1 and 50, 1 and 20, 1 and 10, 5 and 300, 5 and 250, 5 and 200, 5 and 150, 5 and 100, 5 and 50, 5 and 20, 5 and 10, 10 and 300, 20 and 300, 30 and 300, 40 and 300, 50 and 300, 100 and 300, 200 and 300, 50 and 200, 5 and 100, 45 and 125, 25 and 100, 50 and 100, or 100 and 200 mg/ml, e.g., about 5, 10, 15, 20, 25, 30, 35, 40, 45, 50, 60, 70, 80, 90, 100, 150, 200, 250 or 300 mg/ml.

Typically, in therapeutic applications, the treatment would be for the duration of the brain or CNS cancer, and may be continued for maintenance.

Further, it will be apparent to one of ordinary skill in the art that the optimal quantity and spacing of individual dosages will be determined by the nature and extent of the cancer being treated, the form, route and site of administration, and the nature of the particular individual being treated. Also, such optimum conditions can be determined by conventional techniques.

It will also be apparent to one of ordinary skill in the art that the optimal course of treatment, such as, the number of doses of the composition given per day for a defined number of days, can be ascertained by those skilled in the art using conventional course of treatment determination tests.

Those skilled in the art will appreciate that in accordance with the presently disclosed methods and uses the composition may be administered alone or in conjunction with one or more additional agents as part of a combination chemotherapy. For example, the composition may be administered together with one or more additional compounds capable of decreasing cell proliferation and invasion and increasing apoptosis in cancer.

In some embodiments, methods of treatment and/or prevention according to the present disclosure may be applied in combination with a standard chemotherapeutic agent. The chemotherapeutic agent may be selected from, for example: an oxaliplatin, a cisplatin an anthracycline, a taxane, and/or a camptothecin. In some embodiments, chemotherapeutic agents may be an EGFR inhibitor. In some embodiments, chemotherapeutic agents may be immune checkpoint inhibitors. In some embodiments, chemotherapeutic agents may be an immunomodulatory agent (for example, one or more of Atezolizumab, Avelumab Ipilimumab, Bevacizumab, Cemiplimab, Durvalumab, Pembrolizumab, Nivolumab and/or Pidilizumab). In some embodiments, the chemotherapeutic agent is selected from: temozolomide, dacarbazine, carmustine (BCNU), lomustine (CCNU), paclitaxel, docetaxel, vincristine, vinblastine, vinorelbine, etoposide, teniposide, topotecan, irinotecan, doxorubicin, daunomycin, epirubicin, idarubicin, methotrexate, cytarabine, gemcitibine, capecitabine, cisplatin, carboplatin, cyclophosphamide, oxaliplatin, or a mixture thereof.

For such combination therapies, each component of the combination therapy may be administered sequentially in any order, at the same time or at different times, so as to provide the desired effect. Alternatively, the chemotherapeutic agent may be formulated to be administered in the same formulation as the composition.

Therapeutic advantages of the presently disclosed methods and uses may also be realised through combination regimens in conjunction with conventional therapy, such as radiotherapy, chemotherapy, surgery, or other forms of medical intervention.

In some embodiments, the conventional therapy is radiotherapy, which may be administered with or without temozolomide or any other chemotherapeutic agent, such as those described above. The temozolomide or any other chemotherapeutic agent may be administered sequentially in any order, at the same time or at different times as the composition, so as to provide the desired effect. Alternatively, the temozolomide or any other chemotherapeutic agent may be formulated to be administered in the same formulation as the composition.

### Methods of Synthesis

The compositions for use in the methods described herein may be formulated using appropriate methods known in the art.

In one embodiment, the compositions of the present disclosure may be prepared by mixing solid 5-FU (e.g., 15 mg), saline (e.g., 0.9% w/v, 0.87 ml), sodium hydroxide solution (e.g., 0.9 M, 30 μL) and a cyclodextrin, (e.g., β-cyclodextrin sulfated sodium salt as a mixture of 9-12 sulfates per cyclodextrin residue: 45 mg). A resulting suspension may be sonicated at about 30 to 60° C. until almost all of the 5-FU has dissolved. A solution of folinic acid (for example, calcium folinate (1 mg: 10 mg/mL) in 0.9% w/v saline may then added, and the mixture can be subjected to sonication at 30 to 60° C. until a homogenous solution is formed. When sonication is used the temperature of the sonication may be between about 30 and 50, 30 and 40, 40 and 60, 40 and 60 or 40 and 50° C., e.g., about 30, 35, 40, 45, 50, 55 or 60° C., or may be above 60° C. or below 30° C.

A homogeneous, or substantially homogenous, solution may be cooled and allowed to stand at room temperature, for example for about 1 hour. The solution may then be filtered under aseptic conditions and stored at room temperature in the absence of light. The pH of the solution may be between about 7.3 and about 7.5.

Alternatively, the compositions of the present disclosure may be prepared by dissolving a cyclodextrin (e.g., β-cyclodextrin sulfated sodium salt or 2-hydroxypropyl-β-cyclodextrin) in water, and adding a solution of 5-FU (for example 5-fluorouracil Injection BP, 50 mg/mL) and a solution of folinic acid (for example about 200 μL of a 10 mg/mL solution). Thus 300 μL of the above solution may be used per mL of formulation to give a final concentration of 15 mg/mL of 5-FU. The resulting solution is agitated to facilitate mixing. Acid (for example HCl) may then be added if necessary to bring the pH to 7.4±0.1. The solution may then be filtered under aseptic conditions and stored at room temperature in the absence of light.

In one embodiment, a suitable process for making the composition used in the presently disclosed methods and used comprises combining:
(i) 5-FU or a pharmaceutically acceptable salt or analogue thereof;
(ii) folinic acid, or a pharmaceutically-acceptable salt thereof; and
(iii) a cyclodextrin or a pharmaceutically acceptable salt or derivative thereof,
   wherein the concentrations and identities of the components may be as described below.

The process may comprise, for example, combining (i) with (iii), and then admixing (ii). Alternatively, the process may comprise, for example, combining (ii) with (iii), and then admixing the resulting mixture with (i). When admixing (ii), (ii) may be in solution, optionally in aqueous solution. Unless dictated specifically or by context, for a process disclosed herein, components (i), (ii) and (iii) may be mixed in any order. In addition, unless dictated specifically or by context, further components may be added or introduced before, during or after any step comprising one or more of components (i), (ii), and (iii).

The process may also comprise sterilising the composition. The sterilising may comprise filtration (e.g., microfiltration), UV radiation, gamma radiation, some other form of sterilising radiation or some other form of sterilising. The sterilising may be conducted so as not to damage or degrade any of components (i) to (iii) above. Thus sterilising radiation (UV, gamma or other), if used, should be in a dose sufficiently high to sterilise the composition but sufficiently low to avoid damage or degradation of the components specified.

In an embodiment, (i) and (iii) are combined in the presence of a carrier, optionally an aqueous carrier, to form a mixture, and (ii) is admixed with the mixture. The mixture may be a solution, optionally an aqueous solution. The carrier may be a solvent, optionally an aqueous solvent e.g., saline solution, (i) and (iii) may, independently, be partially soluble in the carrier, optionally sparingly soluble. Following combination of (i) and (iii) in the presence of the carrier to form a mixture, the mixture may be agitated to at least partially dissolve (i) and (iii) in the carrier. The dissolution may comprise formation of a non-covalent complex between (i) and (iii). The agitation may comprise stirring, swirling, shaking, sonicating or a combination of two or more thereof. The agitation may be conducted at elevated temperature, e.g., between about 30 and about 60° C. The composition may be agitated following addition of (ii). The agitation may be as described above.

In another embodiment there is provided a process for making a composition, comprising:
combining 5-FU, or a pharmaceutically acceptable salt or analogue thereof, and a cyclodextrin or a pharmaceutically acceptable salt or derivative thereof in the presence of an aqueous solvent to form a mixture;
optionally agitating, and optionally heating, the mixture to form a solution; and
adding an aqueous solution of folinic acid, or a pharmaceutically acceptable salt thereof, to the solution to form the composition.

The step of adding the aqueous solution may be conducted so as to form a composition in which the 5-FU or salt or analogue thereof, the cyclodextrin or salt or derivative thereof and the folinic acid or salt thereof are all in solution in the composition, optionally such that no solid matter is present the composition. The composition may be optionally agitated, and optionally heated, following the step of adding the aqueous solution. The agitation of the composition may be sufficient to render the composition homogeneous or to ensure that all of the components of the composition are completely in solution.

In another embodiment there is provided a process for making a composition, comprising:
combining 5-FU or a pharmaceutically acceptable salt or analogue thereof, and a cyclodextrin or a pharmaceutically acceptable salt or derivative thereof in the presence of an aqueous solvent to form a mixture;
optionally agitating, and optionally heating, the mixture to form a solution;
adding an aqueous solution of folinic acid, or of a pharmaceutically acceptable salt thereof, to the solution to form the composition;
agitating, and optionally heating, the composition sufficient to ensure that all components are in solution; and
sterilising the composition.

In another embodiment there is provided a process for making a composition, comprising:
combining 5-FU, or a pharmaceutically acceptable salt or analogue thereof, in an amount of between about 5 and about 50 mg per ml of final composition, and a cyclodextrin or a pharmaceutically acceptable salt or derivative thereof in an amount of between about 20 and about 200 mg per ml of final composition in the presence of an aqueous solvent to form a mixture;
optionally agitating, and optionally heating, the mixture to form a solution;
adding an aqueous solution of folinic acid, or of a pharmaceutically acceptable salt thereof, at a concentration of between about 5 and 20 mg/ml and in an amount of between about 50 and about 200 μL per ml of final composition, to the solution to form the composition;
agitating, and optionally heating, the composition sufficient to ensure that all components are in solution; and
sterilising the composition.

### Methods of Treatment

The present disclosure relates to the treatment and/or prevention of a cancer of the brain and/or central nervous system (CNS) in a patient, by administering to the patient a therapeutically effective amount of composition comprising:
(i) 5-fluorouracil (5-FU), a pharmaceutically acceptable salt thereof, or a pharmaceutically acceptable analogue thereof;
(ii) folinic acid, or a pharmaceutically acceptable salt thereof; and
(iii) a cyclodextrin, or a pharmaceutically acceptable salt thereof.
   Also disclosed herein is a method of reducing the size of, or slowing the growth of, brain neoplasms in a subject in need of such treatment, comprising administering to the subject an effective amount of a composition comprising:
(i) 5-fluorouracil (5-FU) or an analogue or a pharmaceutically acceptable salt thereof;
(ii) folinic acid, or a pharmaceutically acceptable salt thereof; and
(iii) a cyclodextrin, or a pharmaceutically acceptable salt thereof.

As used herein, “brain” is defined broadly and synonymously to “central nervous system”, to include all cells and tissue of the brain proper and spinal cord of a vertebrate. Thus, the term “brain” includes, but is not limited to, neuronal cells, glial cells, astrocytes, cerebrospinal fluid (CSF), interstitial spaces, brain tunicas, bone, cartilage and the like. The “cranial cavity” refers to any area underneath the skull (cranium), and “intracranial” refers to as being delivered or provided directly into any part of the cranial cavity, except for vascular lumen. Any means known in the art for such direct administration to the brain, e.g., via injection, infusion, are suitable to practice the invention.

Cancers which the methods of the present disclosure are useful for treating and/or preventing include brain tumours and other neoplasia in or around the brain, including both primary tumours and/or metastases that develop in or around the brain. It may also mean metastases of brain tumours that migrate elsewhere in the body. Many types of such tumours and neoplasia are known. Primary brain tumours include glioma, meningioma, neurinoma, pituitary adenoma, medulloblastoma, craniopharyngioma, hemangioma, epidermoid, sarcoma and others. Fifty percent of all intracranial tumours are intracranial metastasis. As used herein, tumours and neoplasia may be associated with the brain and neural tissue, or they may be associated with the meninges, skull, vasculature or any other tissue of the head or neck. Such tumours are generally solid tumours, or they are diffuse tumours with accumulations localized to the head. Tumours or neoplasia for treatment according to the invention may be malignant or benign, and may have been treated previously with chemotherapy, radiation and/or other treatments.

In particular, the methods of the present disclosure are suitable for preventing, treating and/or managing brain and nervous system cancers, including, but not limited to, gliomas, glioblastomas, glioblastoma multiforme, oligodendrogliomas, primitive neuroectodermal tumours, low, mid and high grade astrocytomas, ependymomas (e.g., myxopapillary ependymoma papillary ependymoma, subependymoma, anaplastic ependymoma), oligodendrogliomas, medulloblastomas, meningiomas, pituitary adenomas, neuroblastomas, neurofibromas, malignant peripheral nerve sheath tumours, schwannomas, and craniopharyngiomas.

Exemplary brain cancers include, but are not limited to, gliomas (including astrocytoma (e.g., pilocytic astrocytoma, diffuse astrocytoma, and anaplastic astrocytoma), glioblastoma, oligodendroglioma, brain stem glioma, non-brain stem glioma, ependymoma, and mixed tumours comprising more than one glial cell types. In some embodiments, the suitable cancers include central nervous system tumours including primary CNS tumours such as glioblastomas, astrocytomas (e.g., glioblastoma multiforme) and ependymomas, and secondary CNS tumours (i.e., metastases to the central nervous system of tumours originating outside of the central nervous system). In some embodiments, the brain tumour is a glioma, brainstem glioma, cerebellar or cerebral astrocytoma (e.g., pilocytic astrocytoma, diffuse astrocytoma, or anaplastic (malignant) Astrocytoma), malignant glioma, ependymoma, oligodendrioma, meningioma, craniopharyngioma, hemangioblastoma, medulloblastoma, supratentorial primordial neuroectodermal tumour, visual tract and hypothalamus Gliomas or glioblastomas. In some embodiments, the brain tumour is a glioblastoma (also called glioblastoma multiforme or grade 4 astrocytoma). In some embodiments, the glioblastoma is radiation resistant. In some embodiments, the glioblastoma is radiosensitive. In some embodiments, the glioblastoma can be a substantive glioblastoma. In some embodiments, the glioblastoma is a supratentorial glioblastoma.

The glioma can be any tumour that arises from the glia tissue of the brain. In some embodiments the glioma can be an ependymoma, astrocytoma, oligodendroglioma, brainstem glioma, optic nerve glioma, gliomatosis cerbri, or mixed glioma.

In one embodiment, provided herein is a method of treating and/or preventing ependymomas.

Ependymomas can arise from the ependymal cells that line the brain and the centre of the spinal cord. Ependymomas include, but are not limited to, subependymomas (grade I), myxopapillary ependymomas (grade I), ependymomas (grade II), and anaplastic ependymomas (grade III). Ependymomas (grade II) include, but are not limited to, cellular ependymomas, papillary ependymomas, clear cell ependymomas, and tancytic ependymomas.

In some such embodiments, the ependymoma is a paediatric ependymoma.

In some embodiments, the nervous system cancer to be treated and/or prevented is glioblastoma. In some embodiments, the glioblastoma is a glioblastoma multiforme or grade 4 astrocytoma. In some embodiments, the glioblastoma is radiation resistant. In some embodiments, the glioblastoma is radiosensitive. In some embodiments, the glioblastoma can be a substantive glioblastoma. In some embodiments, the glioblastoma is a supratentorial glioblastoma.

In some embodiments, provided herein are methods for treating and/or preventing relapsed or refractory brain or CNS tumours. In one embodiment, provided herein are methods for treating and/or preventing metastases to the central nervous system of tumours originating outside of the CNS.

### Methods of Administration

In some applications, a composition defined herein (or medicament manufactured from a composition defined herein), is administered to a patient already suffering from cancer, in an amount sufficient to cure, or at least partially arrest the cancer and its complications. For example, the compositions should provide a quantity of: (i) 5-FU, a pharmaceutically acceptable salt thereof, or a pharmaceutically acceptable analogue thereof; and (ii) folinic acid, or a pharmaceutically acceptable salt thereof, sufficient to effectively treat the cancer.

In some embodiments, the methods disclosed herein also include prophylactic or preventative application of the compositions. In some embodiments, the compositions are used as a maintenance therapy.

In one embodiment, depending on the disease to be treated and/or prevented, and the subject's condition, the composition may be administered by oral, parenteral (e.g., intramuscular, intraperitoneal, intravenous, CIV, intracistemal injection or infusion, subcutaneous injection), inhalation, nasal, vaginal, rectal, sublingual, or topical (e.g., transdermal) routes of administration. The composition may be formulated alone or together with one or more active agent(s), in suitable dosage unit with pharmaceutically acceptable excipients, carriers, adjuvants and vehicles, appropriate for each route of administration.

In some preferred embodiments, the composition is administered by e.g., intravenous (IV) or subcutaneous routes. In one embodiment, the composition is administered intravenously; for example, by infusion or injection into a blood vessel. In some embodiments, the composition is administered via a bolus IV and/or an IV infusion. In some embodiments, the IV infusion is a continuous IV infusion.

In one embodiment, the amount of the composition administered in the methods provided herein may range, e.g., between about 5 mg/day and about 2,000 mg/day. Example ranges include between: about 10 mg/day and about 2,000 mg/day, about 20 mg/day and about 2,000 mg/day, about 50 mg/day and about 1,000 mg/day, about 100 mg/day and about 1,000 mg/day, about 100 mg/day and about 500 mg/day, about 150 mg/day and about 500 mg/day, or about 150 mg/day and about 250 mg/day. In certain embodiments, particular dosages are about, or up to about: 10 mg/day, 20 mg/day, 50 mg/day, 75 mg/day, 100 mg/day, 120 mg/day, 150 mg/day, 200 mg/day, 250 mg/day, 300 mg/day, 350 mg/day, 400 mg/day, 450 mg/day, 500 mg/day, 600 mg/day, 700 mg/day, 800 mg/day, 900 mg/day, 1,000 mg/day, 1,200 mg/day, or 1,500 mg/day.

In one embodiment, the amount of the composition in the pharmaceutical composition or dosage form provided herein may range, e.g., between: about 5 mg and about 2,000 mg, 10 mg and about 2,000 mg, about 20 mg and about 2,000 mg, about 50 mg and about 1,000 mg, about 50 mg and about 500 mg, about 50 mg and about 250 mg, about 100 mg and about 500 mg, about 150 mg and about 500 mg, or about 150 mg and about 250 mg. In certain embodiments, particular dosages are about, or up to about: 10 mg/day, 20 mg/day, 50 mg/day, 75 mg/day, 100 mg/day, 120 mg/day, 150 mg/day, 200 mg/day, 250 mg/day, 300 mg/day, 350 mg/day, 400 mg/day, 450 mg/day, 500 mg/day, 600 mg/day, 700 mg/day, 800 mg/day, 900 mg/day, 1,000 mg/day, 1,200 mg/day, or 1,500 mg/day.

In one embodiment, the composition can be delivered as a single dose such as, e.g., a single bolus injection, or oral tablets or pills; or over time such as, e.g., continuous infusion over time or divided bolus doses over time. In one embodiment, the composition can be administered repetitively if necessary, for example, until the patient experiences stable disease or regression, or until the patient experiences disease progression or unacceptable toxicity. Stable disease or lack thereof is determined by methods known in the art such as evaluation of patient's symptoms, physical examination, visualization of the tumour that has been imaged using X-ray, CAT, PET, or MRI scan and other commonly accepted evaluation modalities.

In certain embodiments, the composition is administered to a patient in cycles (e.g., daily administration for one week, then a rest period with no administration for up to three weeks). Cycling therapy involves the administration of an active agent for a period of time, followed by a rest for a period of time, and repeating this sequential administration. Cycling therapy can reduce the development of resistance, avoid or reduce the side effects, and/or improves the efficacy of the treatment.

In one embodiment, a method provided herein comprises administering the composition in 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, or greater than 40 cycles. In one embodiment, the median number of cycles administered in a group of patients is about, or at least about: 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, or 40.

In certain embodiments, treatment cycles comprise multiple doses of the composition administered to a subject in need thereof over multiple days (e.g., 1, 2, 3, 4, 5, 6, 7, 8, 9, 10, 11, 12, 13, 14, or greater than 14 days), optionally followed by treatment dosing holidays (e.g., 1, 2, 3, 4, 5, 6, 7, 8, 9, 10, 11, 12, 13, 14, 15, 16, 17, 18, 19, 20, 21, 22, 23, 24, 25, 26, 27, 28, or greater than 28 days).

In certain embodiments, treatment cycles comprise multiple doses of the composition administered to a subject in need thereof over multiple days (e.g., 1, 2, 3, 4, 5, 6, 7, 8, 9, 10, 11, 12, 13, 14, or greater than 14 days), optionally followed by treatment dosing holidays (e.g., 1, 2, 3, 4, 5, 6, 7, 8, 9, 10, 11, 12, 13, 14, 15, 16, 17, 18, 19, 20, 21, 22, 23, 24, 25, 26, 27, 28, or greater than 28 days). Suitable dosage amounts for the methods provided herein include, e.g., therapeutically effective amounts and prophylactically effective amounts. In certain embodiments, the amount of the composition administered in the methods provided herein may range, e.g., between about 50 mg/m²/day and about 2,000 mg/m²/day, for example between: about 100 mg/m²/day and about 1,000 mg/m²/day, about 100 mg/m²/day and about 500 mg/m²/day, about 50 mg/m²/day and about 500 mg/m²/day, about 50 mg/m²/day and about 200 mg/m²/day, about 50 mg/m²/day and about 100 mg/m²/day, about 50 mg/m²/day and about 75 mg/m²/day, or about 120 mg/m²/day and about 250 mg/m²/day. In certain embodiments, the particular dosage is about, or up to about: 50, 60, 75, 80, 100, 120, 140, 150, 180, 200, 220, 240, 250, 260, 280, 300, 320, 350, 380, 400, 450, or 500 mg/m²/day.

Certain embodiments herein provide co-administration of the composition with one or more additional active agents to provide a synergistic therapeutic effect in subjects in need thereof. The co-administered active agent(s) may be cancer therapeutic agents, such as agents capable of decreasing cell proliferation and invasion and increasing apoptosis in cancer. In certain embodiments, the co-administered active agent(s) may be selected from, for example, an oxaliplatin, a cisplatin an anthracycline, a taxane, and/or a camptothecin. In some embodiments, chemotherapeutic agents may be an EGFR inhibitor. In some embodiments, chemotherapeutic agents may be an immunomodulatory agent. In some embodiments, the chemotherapeutic agent is selected from temozolomide, dacarbazine, carmustine (BCNU), lomustine (CCNU), paclitaxel, docetaxel, vincristine, vinblastine, vinorelbine, etoposide, teniposide, topotecan, irinotecan, doxorubicin, daunomycin, epirubicin, idarubicin, methotrexate, cytarabine, gemcitibine, capecitabine, cisplatin, carboplatin, cyclophosphamide, oxaliplatin, or a mixture thereof.

In one embodiment, a composition as disclosed herein may be administered together with at least one other chemotherapeutic agent as part of a combination chemotherapy regimen, for example at least one chemotherapeutic agent selected from: vincristine, etoposide, cyclophosphamide, cisplatin, carboplatin, and methotrexate (optionally in a high dose). Alternatively, a composition as disclosed herein may be administered together with all-trans retinoic acid (Tretinoin), as part of a combination chemotherapy regimen. In addition to any drug regimen, comprising one or more different drugs and/or doses of the same or different drugs, radiotherapy may be used as part of any treatment.

For such co-administration, the composition may be administered sequentially in any order, at the same time or at different times, with an additional chemotherapeutic agent so as to provide the desired effect. When administered separately, it may be preferred for the components to be administered by the same route of administration, although it is not necessary for this to be so.

Therapeutic advantages of the presently disclosed methods and uses may also be realised through combination regimens in conjunction with conventional therapy, such as radiotherapy, surgery, or other forms of medical intervention.

In some embodiments, the conventional therapy is radiotherapy, which may be administered with or without temozolomide or any other chemotherapeutic agent, such as those described above. The temozolomide or any other chemotherapeutic agent may be administered sequentially in any order, at the same time or at different times as the composition, so as to provide the desired effect. Alternatively, the temozolomide or any other chemotherapeutic agent may be formulated to be administered in the same formulation as the composition.

### EXAMPLES

The present disclosure will now be described with reference to the following non-limiting examples and with reference to the accompanying Figures.

### Example 1—Pharmaceutical Compositions

### (i) Preparation of Compositions

Preparation of a Composition from Solid 5-FU

To make 1 mL of composition: 5-FU (15 mg), 0.9% saline (870 μL), NaOH (30 μL of 0.9 M) and O-cyclodextrin sulfated sodium salt (45 mg) were mixed and the resulting suspension was sonicated at 40-50° C. until almost all of the 5-FU had dissolved. Folinic acid (FA) solution (leucovorin calcium 10 mg/mL, 100 μL) was added and the mixture was sonicated at 40-50° C. until a homogeneous solution formed. The mixture was cooled and allowed to stand for 1 hour at room temperature. The pH of this solution was 7.4±0.1. The solution was filtered under aseptic conditions and stored at room temperature protected from light.

Using the same method but increasing the concentration of β-cyclodextrin sulfated sodium salt produced a composition containing 175 mg of β-cyclodextrin sulfated sodium salt.

Preparation of a Composition from a Solution of 5-FU Solution

To make 1 mL of composition: β-cyclodextrin sulfated sodium salt (45 mg) was dissolved in water (525 μL). 5-FU solution (5-fluorouracil injection BP, 50 mg/mL, 300 μL) and 100 μL of FA solution (leucovorin calcium 10 mg/mL) were added to the cyclodextrin solution which was agitated to mix. HCl (75 μL of 1.000 M solution) was added to the solution to bring the pH to 7.4±0.1. The solution was filtered under aseptic conditions and stored at room temperature, protected from light.

Preparation of a Composition from a Solution of 5-FU Solution

To make 1 mL of composition: (2-hydroxypropyl)-β-cyclodextrin (100 mg) was dissolved in water (527 μL). 5-FU solution (5-fluorouracil injection BP, 50 mg/mL, 300 μL) and 100 μL of FA solution (leucovorin calcium 10 mg/mL) were added to the cyclodextrin solution which was agitated to mix. HCl (73 μL of 1.000 M solution) was added to the solution to bring the pH to 7.4±0.1. The solution was filtered under aseptic conditions and stored at room temperature, protected from light.

Preparation of a Composition from a Solution of 5-FU Solution

To make 1 mL of composition: heptakis(6-O-sulfo)-β-cyclodextrin sodium salt, (50 mg) was dissolved in water (527 μL). 5-FU solution (5-fluorouracil injection BP, 50 mg/mL, 300 μL) and 100 μL of FA solution (leucovorin calcium 10 mg/mL) were added to the cyclodextrin solution which was agitated to mix. HCl (73 μL of 1.000 M solution) was added to the solution to bring the pH to 7.4±0.1. The solution was filtered under aseptic conditions and stored at room temperature, protected from light.

### (ii) Typical Compositions

Some typical compositions for use in the methods of the present disclosure had final concentrations of 5-FU (15 mg/ml) and FA (1 mg/ml), and included hydroxypropyl β-cyclodextrins (e.g., (2-hydroxypropyl)-β-cyclodextrin; 100 mg/ml) or polysulfated β-cyclodextrins (e.g., heptakis(6-O-sulfo)-β-cyclodextrin sodium salt; 45-175 mg/ml). The concentration of the resulting pharmaceutical compositions was expressed in terms of the concentration of 5-FU.

(iii) Pharmaceutical Compositions for Administration

### Injectable Parenteral Composition

A pharmaceutical composition, suitable for administration by injection, may be prepared by mixing 1-5% by weight of the composition of the present disclosure, such as the compositions described above, in 10% by volume propylene glycol and water. The solution is sterilised by filtration. The total amount of the composition may vary from patient to patient although a general dose might be about 750 mg of 5-FU, i.e., 50 mL of formulation that might be diluted with 5% glucose, 0.9% saline or sterile water to a volume of 100, 500, or 1000 mL.

### Composition for Parenteral Administration

For example, a composition for intramuscular injection could be prepared to contain 1 mL sterile buffered water, and 1 mL of the composition of the present disclosure.

Similarly, a composition for intravenous infusion may comprise 250 ml of sterile Ringer's solution, and 5 mL of the composition of the present disclosure.

### Injectable Parenteral Composition

A composition suitable for administration by injection may be prepared by mixing 1% by weight of the composition of the present disclosure by volume propylene glycol and water. The solution is sterilised by filtration.

### Example 2—Preclinical Toxicological Evaluation

Assessment of in vivo toxicity and dose tolerance of the composition used in the methods of the present disclosure was undertaken in rodents.

Accordingly, dose tolerance of test compositions containing a ratio of 15:1 of 5-FU to FA, was compared to that of a comparator treatment with sequential administration of dose-matched 5-FU and FA (‘5-FU:FA’).

The tested compositions had final concentrations of 15 mg/ml 5-FU, 1 mg/ml FA, and comprised either 2-hydroxypropyl-β-cyclodextrin (100 mg/ml final concentration, ‘HP-composition’) or heptakis(6-O-sulfo)-β-cyclodextrin sodium salt (45-175 mg/ml final concentration, ‘S-composition’).

SPF-bred Balb/c mice, Balb/c nu/nu mice and Sprague-Dawley rats were obtained from the Animal Resources Centre (Canning Vale, Western Australia, Australia) or from the University of Adelaide (Urrbrae, South Australia, Australia). All the experiments were approved by the University of Wollongong (UOW) Animal Ethics Committee, the University of Queensland Animal Experimentation Committee, or the Animal Ethics Committee of the University of Adelaide.

Doses administered to animals are stated as mg/m² to simplify comparison with human doses. These were converted to mg/kg doses using body surface area calculation as described (Freireich et al., Cancer Chemother Rep 1966, 219-244). In brief, to convert mg/m² to mg/kg for mice, rats, and rabbits, divide by 3, 5.9 and 12, respectively.

### Intraperitoneal Bolus Assessment

In brief, single or multiple (fractionated) intraperitoneal bolus (i.p.b.) doses of the formulations were compared with the sequential 5-FU:FA treatment and phosphate buffered saline (PBS, 150 mmol/l NaCl solution, pH 7.4) controls in female Balb/c mice. These dose schedules were based on established clinical protocols and take into account shorter murine life expectancy. 5-FU:FA treatments were administered by two separate sequential i.p.b. injections on alternate sides of the midline, with FA being administered immediately before 5-FU. Dose-limiting toxicity endpoints were defined as 15% loss of body weight (compared with the first day of treatment and sustained for >24 hours), or clinical signs of morbidity (i.e. loss of appetite, activity and/or hunched posture, piloerection, and changes in gait).

For some experiments, excised livers, kidneys, and spleens were weighed before fixation and sectioning for haematoxylin and eosin staining and blinded histopathological evaluation. In addition, a proportion of the livers were submitted for oil red-O stains, which is used to identify exogenous or endogenous lipoid deposits.

### Intravenous Bolus and Infusional Assessment

Single intravenous bolus (i.v.b.) doses of the tested compositions were administered to female rats through the peripheral tail vein at dose levels of 425, 475 or 525 mg/m². The positive control group was administered a single i.v.b. dose of FA (35 mg/m²) followed by 5-FU (525 mg/m²) within 20 minutes. Both the treatment groups were then monitored over a 7-day recovery period. In other experiments using both male and female rats, i.v.b. doses of 400 mg/m² of the compositions were followed immediately by continuous intravenous infusion (i.v.i.) of either 600, 1200, or 2400 mg/m² of the compositions over 48 hours with a subsequent 7-day recovery period. The positive control group was matched to the highest tolerated dose of the composition, except that this group also received a single i.v.b. dose of FA (26.7 mg/m², i.e., 1/15 of the single bolus 5-FU dose) immediately before 5-FU administration. Other controls included volume-matched administration of β-cyclodextrin vehicle (pH 7.3) and PBS at pH 7.3 and pH 9 (as a 5-FU pH-matched control).

Clinical signs, systolic blood pressure, proteinuria, and body weights were recorded periodically during the treatment and recovery period. Animals were then euthanised and necropsy performed in all the groups in a blinded manner by a veterinary pathologist. Major organs were also weighed and where indicated, blood was taken to assess haematology. Dose-limiting toxicity endpoints were defined as described above.

### Results

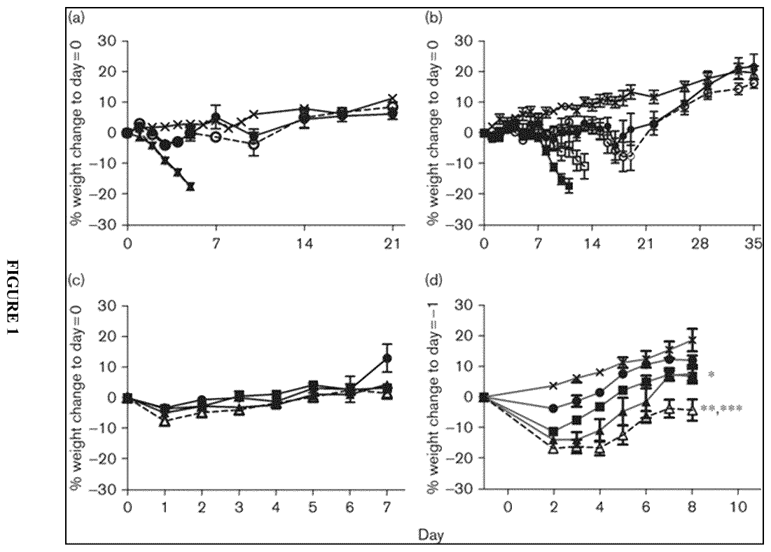

The results are shown in FIG. 1, wherein:
Image (a) relates to the single intraperitoneal bolus (i.p.b.) dose administration in mice. Pooled saline control (x, black line, n=16), 600 mg/m² of the composition comprising polysulfated β-cyclodextrin derivative (‘S-composition’) (black circle, black line, n=3) and 5-FU:FA (white circle, black dashed line, n=3), 675 mg/m² S-composition (black square, black line, n=6) and 5-FU:FA (white square, black dashed line, n=6).
Image (b) relates to multiple/fractionated i.p.b dose administration in mice (n=6 for all cohorts). Saline control (x, black line), 120 mg/m²×5 S-composition (black circle, black line) and 5-FU:FA (white circle, black dashed line), 180 mg/m²×5 S-composition (black square, black line) and 5-FU:FA (white square, black dashed line).
Image (c) relates to single intravenous bolus (i.v.b.) dose administration in rats (n=6 for all cohorts). 425 mg/m² (black circle, black line), 475 mg/m² (black square, black line) and 525 mg/m² (black triangle, black line) of the composition comprising hydroxypropyl β-cyclodextrin derivative (‘HP-composition’), and 525 mg/m² 5-FU:FA (white triangle, black dashed line).
Image (d) relates to i.v.b. plus intravenous infusion (over 48 hours) dose administration in rats (n=8 for all cohorts, equal numbers of males and females). Saline control (x, black line), 400+600 mg/m² HP-composition (black circle, black line), 400+1200 mg/m² HP-composition (black square, black line), 400+2400 mg/m² (black triangle, black line) HP-composition, and 400+2400 mg/m² 5-FU:FA (white triangle, black dashed line).
   All values shown are mean±SEM. *P<0.05 for saline control versus HP-composition at the two highest doses. **P<0.01 for saline control versus 5-FU:FA. ***P<0.05 for HP-composition at all doses versus 5-FU:FA.

A dose-dependent increase in toxicity was only observed in animals administered with the compositions or 5-FU:FA treatment as either a single i.p.b. dose of greater than 600 mg/m² or as a fractionated i.p.b. dose of greater than 120 mg/m²×5 (within 14 days) compared with the saline controls (FIGS. 1(a) and (b), data shown for the S-composition only). No damage to the livers, kidneys, or spleens at 7, 14, and 21 days after i.p.b. administration of <=600 mg/m² of the composition or 5-FU:FA was noted by histopathological analysis of hematoxylin and eosin-stained and oil red-O-stained sections (data not shown) confirming the tolerability and hence no observed adverse effect level of the composition at these doses by this route of administration. Five of the six composition-treated and six of the six 5-FU:FA-treated mice reached toxicity endpoints by day 5 of observation after a single i.p.b. administration of 675 mg/m² dose (FIG. 1(a)). The compositions and 5-FU:FA at 180 and 240 mg/m²×5 i.p.b. resulted in at least three of the six animals in each cohort reaching toxicity endpoints before administration of the ultimate (i.e. only 4×180 mg/m² achieved) and penultimate (i.e. 3×240 mg/m² achieved) doses, respectively. Therefore, the maximum tolerated dose of 5-FU administered either as 5-FU:FA or within the tested compositions lied between 600 and 675 mg/m² of 5-FU given either as single dose or fractionated i.p.b. doses over 2 weeks.

Given the above findings and that the human tolerability of i.v.b. administration of 5-FU is approximately 500 mg/mg², escalating-dose i.v.b. administration of the HP composition was compared with the high dose-matched 5-FU:FA positive controls in rats in this range. No overt signs of toxicity, as indicated by weight changes and other clinical signs, were associated with the administration of single i.v.b. doses of 425, 475, and 525 mg/mg² of the composition or with 525 mg/mg² 5-FU (after i.v.b. administration of 35 mg/mg² FA) (FIG. 1(c)). Although the macroscopic tissue analyses found that the 5-FU group exhibited a significantly higher (P<0.05, Fisher exact test) frequency of liver enlargement, cardiac hypertrophy, mesenteric lymph enlargement, and colon/cecum distension when compared with the animals administered the composition at all the doses tested, no abnormal histological changes were observed. Thus, as the composition (and 5-FU) at 525 mg/m² did not cause toxicity in this study, the NOAEL for the composition is 525 mg/m² when administered as a single i.v.b. dose.

Next, the composition was administered to male and female rats as a single i.v.b. dose of 400 mg/m² followed by continuous i.v.i. over 48 hours at doses ranging from 600 to 2400 mg/m² to mimic a commonly used clinical schedule of 5-FU administration. A dose-dependent increase in toxicity was observed in both the male and female rats who were administered the composition, which manifested as a significant reduction in body weight compared with the controls (FIG. 1, image (d)). This was accompanied by an increase in the moderate-to-severe clinical signs and a number of changes in urine parameters during infusion, most of which normalised after infusion, except that persistent proteinuria was most evident in the male rats given the highest dose of the composition. Otherwise, no sex differences were noted. Changes in haematology and organ weights indicated that the composition at the highest dose induced toxicity in the bone marrow and possibly spleen, manifested particularly by reductions in the white cell and platelet counts.

Several volume-matched controls were used in this study apart from the controls with saline buffered to the exact pH of the composition batch used (i.e. pH 7.3) including controls with saline buffered to pH 9 (to match the pH of the 5-FU solution) and hydroxypropyl β-cyclodextrin (pH 7.3). All controls gave similar results confirming that these components of the formulation did not contribute to toxicity (data not shown). Thus, on the basis of all of these parameters, the NOAEL for the compositions with this regimen was an i.v.b. dose of 400 mg/m² followed by an i.v.i dose of 1200 mg/m² over approximately 2 days.

As a comparator, an i.v.b. dose of FA (26.7 mg/m²) followed by an i.v.b. dose of 5-FU (400 mg/m²) and subsequent i.v.i. of 5-FU (2400 mg/m² over 2 days) was assessed. The rats in this treatment cohort lost significantly more weight than all the other the composition cohorts (FIG. 1(d)).

In general, the majority of parameters measured showed that the compositions given at this maximum tolerated dose produced less severe general toxicity, haematological, and urinary changes than 5-FU when administered under a similar dosing regimen.

### Example 3—Evaluation of Toxicity and Pharmacokinetics in Phase I Clinical Studies

It has been investigated whether the composition used in the methods of the present disclosure is safely tolerated in human patients at various concentrations and dosing levels, and that whether the pharmacology of 5-FU within the composition differs from that of native 5-FU.

### First-In-Human Phase I Study

Clinical safety and tolerability, and pharmacokinetics of test compositions containing a ratio of 15:1 of 5-FU to FA, which is similar to standard sequential low-dose 5-FU:FA bolus regimens, were evaluated in both bolus and infusional schedules.

### Patient Eligibility

Patients over the age of 18 with advanced or metastatic malignancies who had exhausted all standard treatments with an Eastern Cooperation Oncology Group performance status 0-2 were enrolled. A life expectancy of >12 weeks, and satisfactory renal, hepatic and haematological function were required. Exclusion criteria included patients with known deficiency of dihydropyrimidine dehydrogenase or a history of severe reactions to 5-FU or fluoropyrimidines, untreated brain metastases, patients who had completed chemotherapy or radiotherapy <4 weeks prior, or who had severe comorbidities. Pregnant or breastfeeding women were also excluded.

### Study Design and Treatment

This open-label single-centre phase I study used a standard 3+3 dose escalation scheme to explore two treatment regimens. The primary objectives were to evaluate the safety and tolerability of the composition in subjects with relapsed or refractory malignancy and to determine the maximum-tolerated dose (MTD). Secondary objectives were 5-FU pharmacokinetic profile. This study was approved by a local Human Investigations Committee (Bellberry Limited Approval #2014-05-259; TGA CTN 2014/0737) and written informed consent was obtained from all patients.

Patients were treated with the test composition (5-FU 15 mg/mL; FA 1 mg/mL; hydroxypropyl β-cyclodextrin 100 mg/mL, pH 7.4±0.1; formulated as ready-to-use solution) either as a bolus [based on the colorectal adjuvant Roswell Park (modified) fluorouracil and leucovorin weekly regimen; ID: 1271 v.4] or continuous infusion [based on the colorectal adjuvant de Gramont (modified) regimen; ID: 76 v.4]. Treatment regimens were allocated based on clinical factors such as the presence of an existing central line. Bolus injections were administered within approximately 5 minutes via a peripheral cannula or central line weekly for 6 consecutive weeks every 8 weeks. Infusional injections were administered continuously over approximately 46 hours via a central line, portacath or PICC line using a CADD Pump every 2 weeks for 12 weeks, followed by a 2 weeks break before patients were eligible for a repeat cycle of treatment. Patients continued the composition while tolerated until disease progression. The dose escalation levels and number of patients enrolled in the bolus and infusion regimens are summarized in Table 1.

TABLE 1
Number of patients and doses received in each treatment schedule (bolus weekly × 6, and 46 hours infusion every 2 weeks × 6)

| | Bolus | | | | | Infusion | | | | |
|---|---|---|---|---|---|---|---|---|---|---|
| | Deflexifol | | | Mean | Treatment | Deflexifol | | | Mean | Treatment |
| | dose | No. | | No. | related and | dose | No. | | No. | related and |
| | (mg/m² 5-FU | patients | No. | doses/ | non-related | (mg/m² 5-FU | patients | No. | doses/ | non-related |
| Dose level | equivalents) | treated | doses | patient | AEs | equivalents) | treated | doses | patient | AEs |
| 1 | 375 | 3 | 35 | 11.67 | no toxicity | 1200 | 3 | 19 | 6.33 | 1 × incomplete |
| 2 | 425 | 3 | 34 | 11.33 | no toxicity | 1800 | 3 | 21 | 7 | bowel obstruction |
| 3 | 475 | 3 | 30 | 10 | 1 × grade 3 | 2400 | 6 | 62 | 10.33 | at dose 3 cycle 1, |
| | | | | | Dyspnoea + | | | | | withdrew consent |
| | | | | | compression | | | | | 1 × ALT/AST |
| | | | | | fracture | | | | | increase due to |
| | | | | | 1 × grade 3 | | | | | unrelated infection, |
| | | | | | diarrhoea | | | | | did not complete |
| | | | | | | | | | | Patient #25 |
| | | | | | | | | | | received >4 |
| | | | | | | | | | | cycles, 12 months |
| | | | | | | | | | | treatment |
| | | | | | | | | | | 1 × SAE fall out of |
| 4 | 525 | 6 | 41 | 6.83 | 1 patient with | 3000 | 6 | 31.5 | 5.25 | bed - attributed to |
| | | | | | suspected DPDD, | | | | | rising Dysnopea, |
| | | | | | did not complete | | | | | related to PD |
| 5 | 575 | 4 | 19 | 3.8 | 1 × grade 3 | 3600 | 3 | 10 | 3.33 | No patients |
| | | | | | diarrhoea | | | | | completed a full |
| | | | | | 1 × grade 3 | | | | | treatment cycle due |
| | | | | | diarrhoea + | | | | | to PD |
| | | | | | myelosuppression | | | | | |
| | Total | 19 | 159 | | | Total | 21 | 143.5 | | |

### Safety Evaluations

All adverse events (AEs) were graded according to the National Cancer Institute Common Terminology Criteria for Adverse Events version V4.03. AE monitoring continued for 4 weeks after the final treatment, and monitoring of AEs related to the composition was continued until stabilization or resolution. Dose-limiting toxicities (DLTs) were assessed as AEs that were possibly related to study treatment, including any grade 3 or 4 nonhematologic toxicity with the exception of grade 3 nausea, vomiting, alopecia or diarrhoea that resolved to a lower grade with supportive treatment within 7 days; febrile neutropenia, grade 4 neutropenia without fever lasting >7 days; grade 4 thrombocytopenia lasting >7 days; any grade of thrombocytopenia associated with bleeding.

In both schedules, MTD was declared as the dose level previous to the one at which two or more patients (out of 6) experienced DLTs.

### Pharmacokinetic Evaluations

Blood samples were taken from patients on both the bolus and infusional regimens during the first and sixth dose of the composition (bolus: pre, 10, 20, 60, 120 minutes and 24 hours; infusion: pre and 2 hours). Blood plasma levels of 5-FU and its metabolite 5-fluoro-5,6-dihydrouracil (5-FUH₂) were measured by HPLC method with minor modifications. Area under the curve (AUC), clearance (CLR) and plasma half-lives (t_{1/2}) were estimated for each patient to assess PK variability and adequacy of dosing in comparison to historical data.

### Statistical Analysis

Descriptive statistics on patient characteristics, analysis of toxicities and outcomes were performed for all patients. The Kaplan and Meier method was used to calculate progression-free and overall survival from the treatment start date to the date of death, or last review. PK values were calculated by program “PK Functions for Microsoft Excel” using add-ins of PK1 and PK2 functions to excel data analysis files, and Statistica (V12) was used for simple descriptive statistics to summarize PK data within each dose level and, where appropriate, across dose levels.

### Patient Characteristics

Forty patients (19 bolus, 21 infusion) were entered into the study. Patients were heavily pre-treated, with 13/40 (33%) of patients having previously undergone more than five lines of treatment and 34/40 (85%) had failed prior fluoropyrimidine treatment. The most common tumour types were colorectal (60%) and breast cancer (18%).

### Results: Safety

No grade 4 AEs were observed in any patients (Table 2). Only eight patients (20%) reported treatment-related AEs with a severity of grade 3. The DLT in the bolus schedule was grade 3 diarrhoea and myelosuppression (pancytopenia) at 575 mg/m² (dose of 5-FU). The bolus regimen MTD is thus 525 mg/m², which exceeds that of current standard colorectal cancer adjuvant or metastatic weekly schedules (e.g., AIO, Roswell Park; 375-500 mg/m²) and with DLTs consistent with that reported in various weekly bolus 5-FU regimens. No DLT was observed in the infusion schedule up to dose level 5 (3600 mg/m²; Table 2). However, it should be noted that no patients at this dose level completed a full treatment cycle due to disease progression, and it was decided to halt the trial at this point and consolidate the dose at 3000 mg/m².

TABLE 2
Treatment-related grade 3 or 4 adverse events by dose level and regimen

| | Bolus dose level | | | | | | | | | | Infusion dose level | | | | | | | | | |
|---|---|---|---|---|---|---|---|---|---|---|---|---|---|---|---|---|---|---|---|---|
| | 1 | | 2 | | 3 | | 4 | | 5 | | 1 | | 2 | | 3 | | 4 | | 5 | |
| | Grade | | | | | | | | | | Grade | | | | | | | | | |
| | No. of events | | | | | | | | | | No. of events | | | | | | | | | |
| Toxicity | G3 | G4 | G3 | G4 | G3 | G4 | G3 | G4 | G3 | G4 | G3 | G4 | G3 | G4 | G3 | G4 | G3 | G4 | G3 | G4 |
| Diarrhoea | 0 | 0 | 0 | 0 | 0 | 0 | 1^{a} | 0 | 2 | 0 | 0 | 0 | 0 | 0 | 1^{b} | 0 | 0 | 0 | 0 | 0 |
| Nausea and | 0 | 0 | 0 | 0 | 0 | 0 | 0 | 0 | 0 | 0 | 0 | 0 | 0 | 0 | 1^{b} | 0 | 0 | 0 | 1 | 0 |
| vomiting | | | | | | | | | | | | | | | | | | | | |
| Dyspnea | 0 | 0 | 0 | 0 | 0 | 0 | 0 | 0 | 0 | 0 | 0 | 0 | 0 | 0 | 0 | 0 | 0 | 0 | 0 | 0 |
| Febrile | 0 | 0 | 0 | 0 | 0 | 0 | 1 | 0 | 0 | 0 | 0 | 0 | 0 | 0 | 0 | 0 | 0 | 0 | 0 | 0 |
| neutropenia | | | | | | | | | | | | | | | | | | | | |
| Pancytopenia | 0 | 0 | 0 | 0 | 0 | 0 | 0 | 0 | 1 | 0 | 0 | 0 | 0 | 0 | 0 | 0 | 0 | 0 | 0 | 0 |
| Venous | 0 | 0 | 0 | 0 | 0 | 0 | 1 | 0 | 0 | 0 | 0 | 0 | 0 | 0 | 0 | 0 | 0 | 0 | 0 | 0 |
| thrombosis | | | | | | | | | | | | | | | | | | | | |
| Raised | 0 | 0 | 0 | 0 | 0 | 0 | 0 | 0 | 0 | 0 | 0 | 0 | 0 | 0 | 0 | 0 | 0 | 0 | 0 | 0 |
| ALT/AST | | | | | | | | | | | | | | | | | | | | |

^{a}Patient No. 40. This AE occurred after the fifth dose of their first cycle and was resolved by changing to a 4 weeks on 2 weeks off schedule. The patient then continued through another two full cycles with no AEs, declining further treatment in their fifth cycle.
^{b}Patient No. 17. These AEs occurred after the third dose of their first cycle and were resolved by 25% dose reduction. The patient then continued on to another cycle with no AEs.

Overall, grade 1-2 fatigue and nausea were the most common toxicities observed among patients in both bolus and infusional regimens. No >grade 2 toxicity was noted at 375-475 mg/m² bolus (dose levels 1-3), or at 1200-1800 mg/m² infusion (dose levels 1-2). No cardiac toxicity was observed. Grade 1-2 myelosuppression was only observed in the bolus regimen. Three grade 3 AEs were observed but these were related to disease progression and not to the study drug.

### Results: Pharmacokinetics

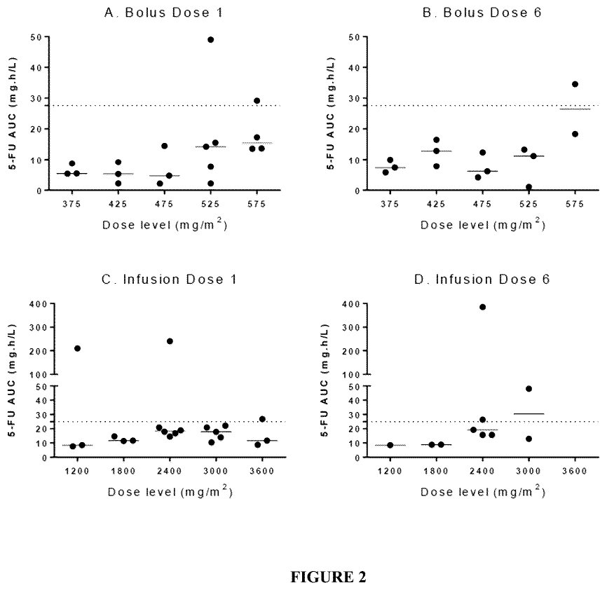

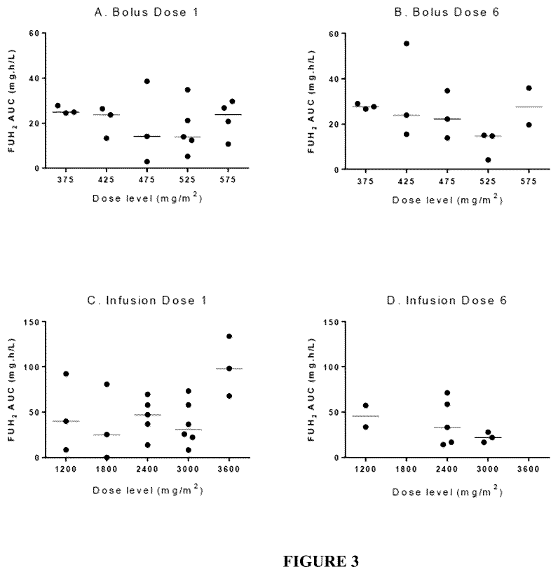

Of the 40 patients available for assessment of PK variability and adequacy of dosing, 38/40 patients treated at dose 1 and 24/32 patients treated at dose 6 had plasma levels assessed. All patients had measurable plasma concentrations of 5-FU and FUH₂, with the FUH₂ levels consistently greater than 5-FU (FIGS. 2 and 3), as expected for patients with normal dihydropyrimidine dehydrogenase activity and 5-FU catabolism.

FIG. 2 shows scatter dot plots show relationship of area under the 5-FU plasma concentration×time curve (AUC) versus dose level (mg/m²) measured after administration of (A, B) bolus dose 1 and 6, or (C, D) during infusion dose 1 and 6. • represents an individual patient with median values (−) for the cohort shown. For infusion dose 6, no samples were collected at the 3600 mg/m² dose level. Dashed lines indicate historical median AUC for toxicity using bolus or infusion regimens.

FIG. 3 shows scatter dot plots show relationship of area under the FUH₂ plasma concentration-x-time curve (AUC) versus dose level (mg/m²) measured during administration of (A, B) bolus dose 1 and 6, or (C, D.) infusion dose 1 and 6. • represents an individual patient with median values (−) for the cohort shown. For infusion dose 6, no samples were collected at 1800 and 3600 mg/m² dose levels.

PK showed evidence of interpatient variability consistent with known pharmacology of 5-FU. In the weekly bolus schedule 5-FU CLR was 21-900 L/h, t₁₂ 0.11-0.52 hours, with the intrapatient dose 6 CLR equal to 54-117% of dose 1 (Table 3) and there was a trend to increased AUC (mg-h/L) with dose (FIG. 2). The first 4 dose levels gave median AUCs that are well below the median AUC for toxicity in a study using a weekly bolus schedule of 500-864 mg/m². With the infusion schedule, 5-FU CLR (13-700 L/h) and AUC estimates were somewhat variable due to three patient outliers (>10-fold AUC values compared to the median; FIG. 2 images C and D) and some cases having insufficient data to thoroughly analyse, especially at dose 6 (Table 4). However, compared to historical PK data of 5-FU alone, AUC was likely sub-therapeutic until >525 mg/m² in the bolus schedule, and for some patients with infusion at all dose cohorts.

TABLE 3
Pharmacokinetic parameters of 5-FU in bolus administration of the composition (mean ± SEM)

| | Dose 1 (cycle 1) | | | | Dose 6 (cycle 1) | | | |
|---|---|---|---|---|---|---|---|---|
| Dose levels | | AUC | CLR | t_{1/2} | | AUC | CLR | t_{1/2} |
| (mg/m²) | N | (mg · h/L) | (L/h) | (h) | N | (mg · h/L) | (L/h) | (h) |
| 1 (375) | 3 | 6.5 ± 1.1 | 97.1 ± 11.2 | | 3 | 7.7 ± 1.2 | 84.7 ± 16.7 | |
| 2 (425) | 3 | 5.5 ± 2.0 | 188.0 ± 76.8 | | 3 | 12.4 ± 2.5 | 68.0 ± 19.4 | |
| 3 (475) | 3 | 7.1 ± 3.8 | 201.8 ± 101.4 | | 3 | 7.5 ± 2.5 | 129.8 ± 42.2 | |
| 4 (525) | 5 | 17.7 ± 8.2 | 148.4 ± 85.0 | | 3 | 8.5 ± 3.8 | 378.3 ± 306.9 | |
| 5 (575) | 4 | 18.4 ± 3.7 | 59.4 ± 9.9 | | 2 | 26.5 ± 8.1 | 37.8 ± 9.6 | |
| All | 18 | 12.2 ± 2.7 | 135.6 ± 31.0 | 0.21 ± 0.02 | 14 | 11.5 ± 2.2 | 147.0 ± 66.1 | 0.22 ± 0.02 |

Abbreviations: AUC, area under the curve; CLR, clearance; t_{1/2}, terminal half-life.

TABLE 4
Pharmacokinetic parameters of 5-FU in infusional
administration of the composition (mean ± SEM)

| | Dose 1 (cycle 1) | | | Dose 6 (cycle 1) | | |
|---|---|---|---|---|---|---|
| Dose levels | | AUC | CLR | | AUC | CLR |
| (mg/m²) | N | (mg · h/L) | (L/h) | N | (mg · h/L) | (L/h) |
| 1 (1200) | 3 | 75.2 ± 67.1 | 191.4 ± 89.2 | 1 | 8.56 | 287.5 |
| 2 (1800) | 3 | 12.6 ± 1.04 | 229.3 ± 10.0 | 2 | 8.81 ± 0.1 | 260.1 ± 61.0 |
| 3 (2400) | 6 | 54.7 ± 37.0 | 209.1 ± 40.4 | 5 | 92.4 ± 73.2 | 183.4 ± 46.5 |
| 4 (3000) | 5 | 17.0 ± 2.2 | 355.3 ± 33.1 | 2 | 30.5 ± 17.6 | 322.2 ± 217.4 |
| 5 (3600) | 3 | 15.7 ± 5.6 | 496.2 ± 131.0 | 0 | — | — |
| All | 20 | 36.2 ± 14.5 | 289.1 ± 34.4 | 10 | 54.9 ± 36.9 | 236.9 ± 44.4 |

Overall, this Phase I study demonstrated that the compositions are safe and tolerable, and can be administered to patients at 5-FU doses higher than those used in current clinical practice. In both a bolus and an infusion schedule, the toxicity spectrum of the compositions is minimal, with no unexpected adverse effects. Pharmacokinetic studies suggested that 5-FU in the compositions is distributed and metabolized similarly to native 5-FU.

### Second Phase I Study

A second ongoing Phase 1 study in human patients at various concentrations and dosing levels has confirmed that the composition used in the methods of the present disclosure is safe and tolerable to at least 525 mg/m² bolus followed by 3000 mg/m² infusion (tolerability at 25% greater dose than standard of care with traditional 5-FU:FA formulations) with similar pharmacokinetic profile to historical 5-FU pharmacokinetic profile data. Further data suggests that an infusion with a dose of 3400 mg/m² is viable (a 42% higher tolerable dose).

Together, the clinical studies have shown safety in bolus and infusion doses of the composition used in the methods of the present disclosure to be higher than that of separate 5-FU and FA infusions.

### Example 4—In Vitro Efficacy in Brain Cancer Cells

Assessment of in vitro activity of the composition used in the methods of the present disclosure was undertaken in patient-derived Diffuse Midline Glioma (DMG) neurospheres.

In particular, activity of the composition against human DMG cells was compared to that of 5-FU, a combination of 5-FU and FA, or a media control.

### Experimental Setup

The composition used in the methods of the present disclosure was tested at a 5-FU to FA (Leucovorin) ratio of 15:1 (termed ‘Deflexifol’), and compared to 5-FU as a single agent (‘5-FU’); FA (Leucovorin) as a single agent (‘Leucovorin’); or a combination of 5-FU and FA (Leucovorin) (‘5-FU+Leucovorin’).

The tested concentrations were selected with reference to the clinical maximum tolerated plasma concentration of 5-FU (530 μM), as detailed in Table 5.

TABLE 5
Efficacy study tested drug compositions

| | |
|---|---|
| (i) Deflexifol | |
| Equal 5-FU concentrations | 50 nM, 500 nM, 5 μM, 50 μM |
| | and 500 μM |
| Equal Leucovorin concentrations | 0.72 nM, 7.2 nM, 72 nM, 720 nM |
| | and 7.2 μM |
| (ii) 5-FU single agent: | |
| Concentrations | 50 nM, 500 nM, 5 μM, 50 μM |
| | and 500 μM |
| (iii) Leucovorin single agent | |
| Concentrations | 0.72 nM, 7.2 nM, 72 nM, 720 nM |
| | and 7.2 μM |
| (iv) 5-FU + Leucovorin | |
| 5-FU concentrations | 50 nM, 500 nM, 5 μM, 50 μM |
| | and 500 μM |
| Leucovorin concentrations | 0.72 nM, 7.2 nM, 72 nM, 720 nM |
| | and 7.2 μM |

Screening was conducted in a 96-well format, with N=4 curves, using:
three different DMG cell lines: P002306, DIPG-7 and RA055
one ependymoma culture and
one set of healthy astrocytes (control model system).

### Results

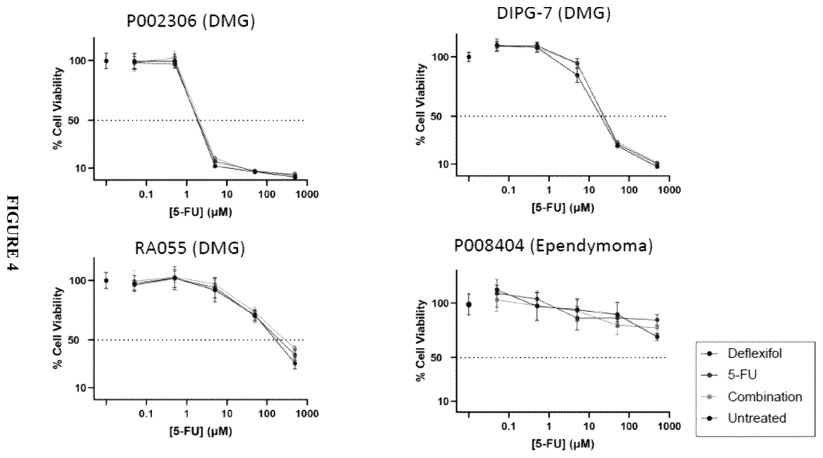

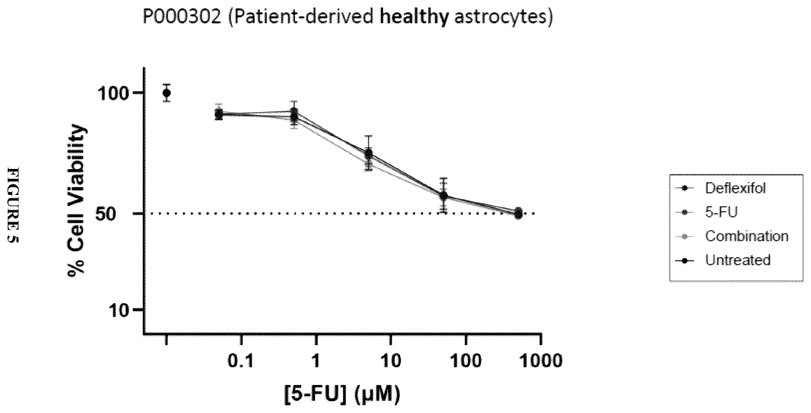

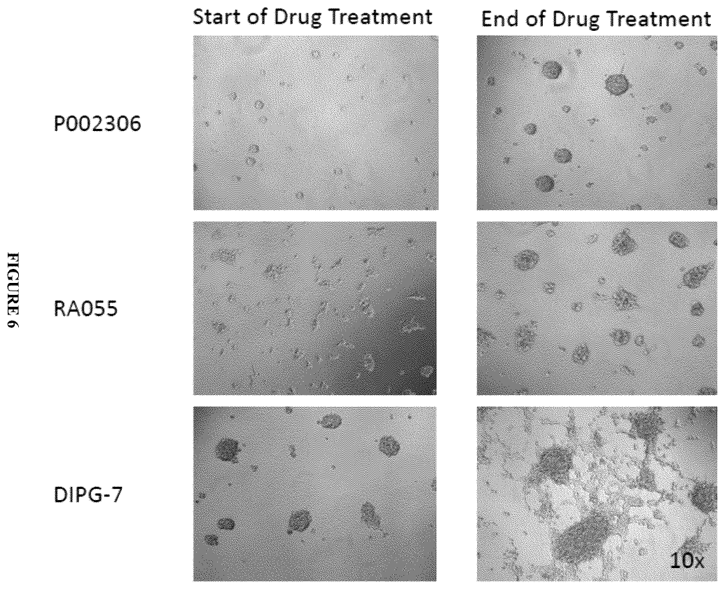

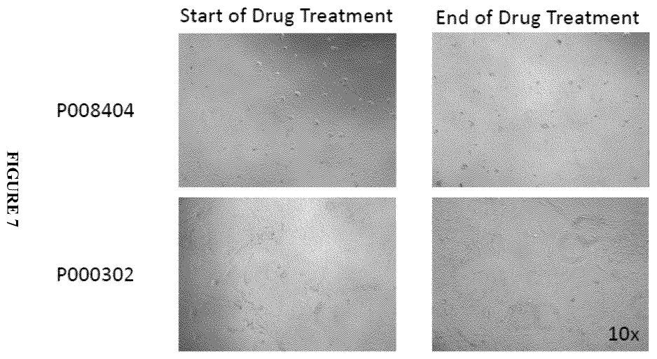

The results are shown in FIGS. 4 and 5 indicate where Deflexifol provides an improved IC₅₀ over 5-FU+Leucovorin, and that the sensitivity of the DMG cells was cell line-dependent: P002306>DIPG-7>RA055> healthy astrocytes>ependymoma. Images showing a comparison of the cells at the start and at the end of the drug treatment are shown in FIGS. 6 and 7.

A summary of dose response values (AUC, IC₅₀ values), is shown in Table 6.

TABLE 6
Dose response values

| Patient | Deflexifol | 5-FU | Combination |
|---|---|---|---|
| P002306 | IC₅₀: ~1.8 μM | IC₅₀: ~1.9 μM | IC₅₀: ~2.1 μM |
| (DMG) | AUC: 169.0 | AUC: 171.4 | AUC: 179.4 |
| RA055 | IC₅₀: ~160 μM | IC₅₀: ~200 μM | IC₅₀: ~290 μM |
| (DMG) | AUC: 329 | AUC: 332.6 | AUC: 345.0 |
| DIPG-7 | IC₅₀: ~18.9 μM | IC₅₀: ~22.3 μM | IC₅₀: ~23.7 μM |
| (DMG) | AUC: 276.3 | AUC: | AUC: 291.2 |
| P008480 | IC₅₀: >500 μM | IC₅₀: >500 μM | IC₅₀: >500 μM |
| (Ependymoma) | AUC: 371.3 | AUC: 373.2 | AUC: 360.3 |
| P000302 | IC₅₀: ~500 μM | IC₅₀: ~500 μM | IC₅₀: ~500 μM |
| (Healthy cells) | AUC: 293.0 | AUC: 294.3 | AUC: 286.5 |

Critically, this data demonstrated that the composition used in the present disclosure has potent anti-cancer activity against DMG cells, whereby the activity varies depending on the cancer cell line. The slow rate of growth of the particular ependymoma culture may require a longer duration of exposure of the cells to Deflexifol.

### Example 5—Pharmacokinetic and Brain Uptake Study of Deflexifol in Female BALB/cJAusb Mice

### Aims

An aim of this study was to assess the uptake of Deflexifol spiked with 5 μCi 6-[³H]5FU into the brains of juvenile BALB/cJAusb mice. A sub-aim was to assess plasma clearance and tissue distribution into the liver and kidneys after intravenous (i.v.) administration of Deflexifol spiked with [³H]5FU as all pre-clinical studies to date have administered Deflexifol via intraperitoneal (i.p.) injection.

### Experimental Methods

### Ethics

AE18/13 approved by the Institutional Animal Ethics Committee on 9 Apr. 2021.

Preparation of Deflexifol Spiked with 6-[³H]5FU
The composition used here was tested at a 5-FU to FA (Leucovorin) ratio of 15:1.
Deflexifol (1500 mg 5-Fluorouracil in 100 ml water containing 100 g cyclodextrin, 10 mg leucovorin; batch No.: DF090421) provided by FivepHusion was spiked with 5-fluorouracil [6-³H] (Moravek, Cat No. MT-686, 1 mCi/mL; Batch No: 592-1 34-0224-A-2021 0428-JPL) on 23 Jun. 2021.

### Mice

Nine 4-5 week old female inbred BALB/cJAusb mice were purchased from Australian Bio Resources (Mossvale, NSW) and arrived at the UOW Animal Facility on Monday 21 Jun. 2021. Mice were allocated into cages of three and housed in isolator cages. Mice were acclimatised for 3 days and received the following tail markings for identification: left (L), right (R) or no marking (N). Mice were weighed on the day of injection.

### Injections Treatments

The study commenced on 24 Jul. 2021. Mice were warmed prior to i.v. injections in the warming cabinet (35° C.) for 30 minutes. Mice were restrained and tails swabbed with ethanol immediately prior to injection of 94 mg/kg Deflexifol spiked with 5 μCi 6-[³H]5FU in 100 μL via the lateral tail vein using 29-gauge insulin needles (AESOP_IV02).
Mice were sacrificed via CO₂ inhalation (AESOP_HK01) at: 10 minutes, 60 minutes or 1440 minutes (24 hours) post injection. Blood was collected via a cardiac puncture and placed into EDTA containing tubes, and the plasma collected. Plasma and organs/tissues were processed as per FivepHusion SOP BD PK tritiated tissue sample processing. Mice were perfused with PBS for ˜2 minutes (AESOP_PF-01), and major organs (brain, kidneys, liver) collected and weighed. The tail was additionally collected and weighed for dose correction.
The treatment identification is shown in Table 7

TABLE 7
Treatment identification

| | | | Time | |
|---|---|---|---|---|
| Cohort | Cage | Identification | (minutes) | Label |
| A | 81321/ | Left Tail Mark | 1440 | 1A7 |
| i.v. DF | 0029 | Right Tail Mark | 1440 | 1A8 |
| | | No Tail Mark | 1440 | 1A9 |
| A | 81321/ | Left Tail Mark | 60 | 1A4 |
| i.v. DF | 0030 | Right Tail Mark | 60 | 1A5 |
| | | No Tail Mark | 60 | 1A6 |
| A | 81321/ | Left Tail Mark | 10 | 1A1 |
| i.v. DF | 0031 | Right Tail Mark | 10 | 1A2 |
| | | No Tail Mark | 10 | 1A3 |

DF: Deflexifol

### Results

### Animal Weight and Dose of Deflexifol

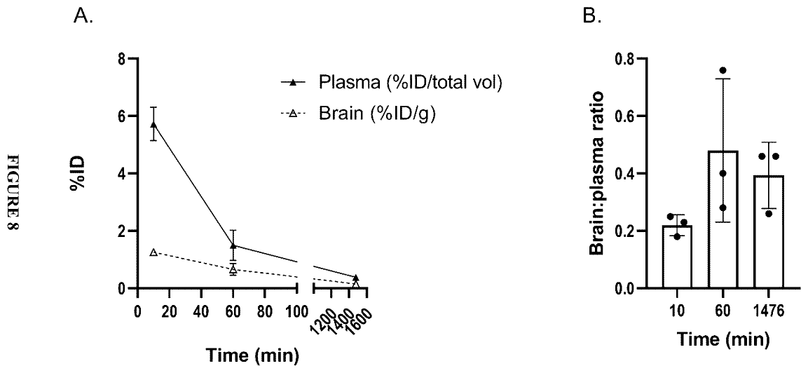

FIG. 8 shows the uptake of the composition used in the presently disclosed methods into mouse brain, represented as (A) percent injected dose (% ID) of the composition in the brain or plasma or (B) brain-to-plasma ratio at indicated time-points. All values are corrected to activity per mL plasma or percent of injected dose and are presented as mean+/−SD (n=3 per time point). Data was analysed using a two-way ANOVA using Graphpad Prism v9.

Radioactivity was detected in the brains within 10 minutes after treatment (range 1.13-1.33% of injected dose/g tissue) concomitant with a rapid loss of tritiated 5-FU from the systemic plasma compartment (FIG. 8,image A). The rapid elimination of 5-FU from the plasma compartment matches historical data for native 5-FU for plasma elimination in rodents, primates and humans.

The brain-to-plasma ratio increased over 1 hour and was maintained at 24 hours suggesting slower elimination of 5-FU from the brain than from plasma (FIG. 8,image B).

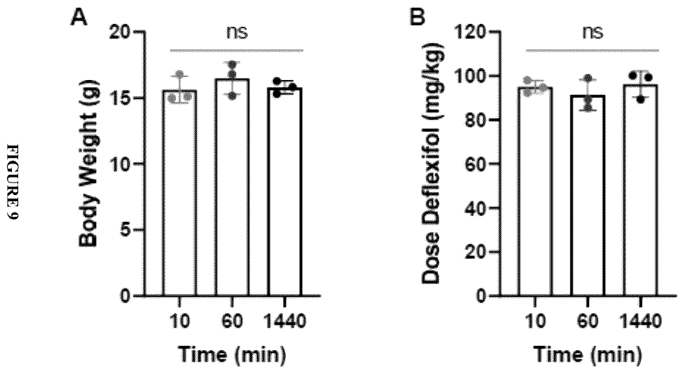

Animal weight was recorded on the day of treatment (FIG. 9, image A). In FIG. 9, the data is presented as mean±SD (n=3). Data was analysed using an ordinary one-way ANOVA using Graphpad Prism v9.

The average weight of mice was 15.97 g±0.92. Minimum weight was 14.98 g and maximum weight was 17.53 (range 2.55 g). The average dose of Deflexifol mice received was 94.2 mg/kg±5.3. The minimum dose delivered was 85.6 mg/kg and the maximum dose delivered was 100.1 mg/kg (FIG. 9, image B). Previous UOW preclinical PK/BD studies have administered 150 mg/kg Deflexifol (FD100HPC) spiked with 2.5 μCi 6-[3H]5FU in 200 μL, i.p. (Stutchbury et al., 2011).

### Organ Weights

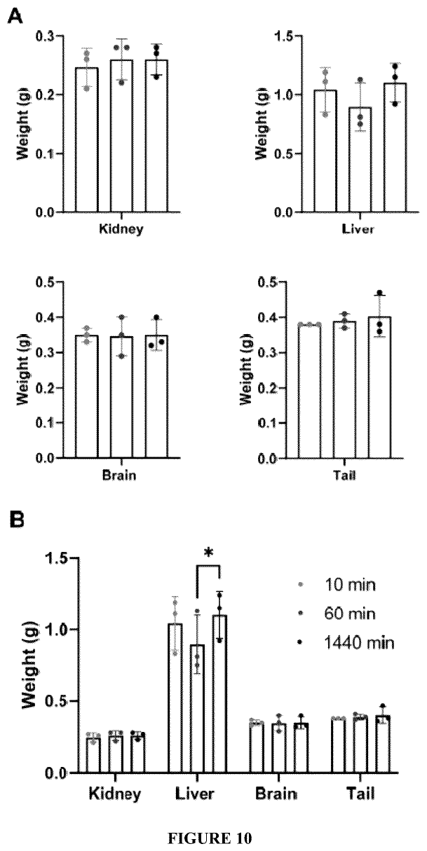

After sacrifice and perfusion, organs were weighed (FIG. 10). In FIG. 10 the data is presented as mean±SD (n=3). Data was analysed using a two-way ANOVA using Graphpad Prism v9.

There was a significant difference in liver weights between the 60 minutes and the 1440 minutes groups (p=0.043) when analysing the data using a two-way ANOVA. The average tissue/organ weight across all cohorts were; kidneys (0.26 g±0.008), liver (1.01 g±0.11), brain (0.35 g±0.002) and tail (0.39 g±0.01).

### Pharmacokinetics and Tissue Distribution

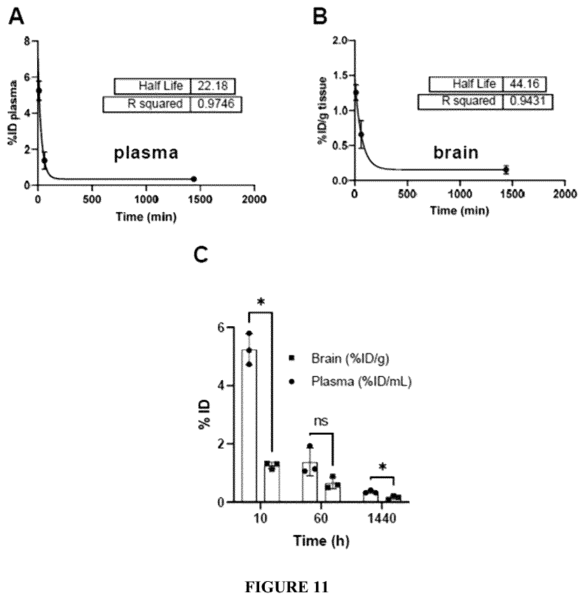

This study primarily sought to assess the uptake of tritiated 5-FU when delivered as Deflexifol into the brains of juvenile mice. The percentage of the injected dose remaining in the brain and plasma at each time point was measured and the clearance of Deflexifol from the plasma and brain was compared by fitting a one-phase decay model (FIG. 11, images A and B). In FIG. 11, data was analysed using a two-way ANOVA using Graphpad Prism v9. In addition, in FIG. 11, all values are corrected to activity per mL plasma (A) or percent of injected dose (B) and are presented as mean+/−SD (n=3 per time point). For FIG. 11, images A and B, data was fitted to a one-phase decay model. FIG. 11, image C shows the % ID in plasma and brain.

The half-life of 5-FU in Deflexifol in the plasma was 22.18 min (FIG. 11, image A). Previously, the half-life of 150 mg/kg Deflexifol (FD100HPC) spiked with 2.5 μCi [6-³H]5FU was determined to be 24.01 minutes when delivered using an i.p. route of administration in female Balb/c mice (Stutchbury et al., 2011). In this study, the half-life of tritiated 5-FU in Deflexifol in the brain was 44.16 min (FIG. 11, image B). The Cₘₐₓ was 1.26% ID/g±0.110 and the Tₘₐₓ was 10 minutes. The area under the curve (AUC) for Deflexifol in the plasma was 1357 (std. error 334.2). The AUC for Deflexifol in the brain was 606.7 (std. error 143.3) after a single bolus dose. A summary of PK data for plasma and brain can be found in Table 8.

TABLE 8
One-phase decay summary data.

| | Plasma (% ID/mL) | Brain (% ID/g) |
|---|---|---|
| One phase decay | | |
| Best-fit values | | |
| Y0 | 7.043 | 1.444 |
| Plateau | 0.3500 | 0.1533 |
| K | 0.03124 | 0.01570 |
| Half Life | 22.18 | 44.16 |
| Tau | 32.01 | 63.71 |
| Span | 6.693 | 1.291 |
| 95% CI | | |
| Y0 | 5.911 to 9.315 | 1.169 to 1.751 |
| Plateau | −0.2388 to 0.9388 | −0.03830 to 0.3450 |
| K | 0.02050 to 0.06286 | 0.007916 to 0.02908 |
| Half Life | 11.03 to 33.82 | 23.84 to 87.56 |
| Tau | 15.91 to 48.79 | 34.39 to 126.3 |
| Goodness of Fit | | |
| Degrees of Freedom | 6 | 6 |
| R squared | 0.9746 | 0.9431 |
| Sum of Squares | 1.042 | 0.1104 |
| Sy · x | 0.4168 | 0.1356 |
| Constraints | | |
| K | K > 0 | K > 0 |
| Number of points | | |
| # of X values | 9 | 9 |
| # Y values analyzed | 9 | 9 |

In order to predict the permeability of a molecule across the blood-brain barrier (BBB), the brain-to-plasma ratio can be used. According to Hitchcock and Pennington (2006), a molecule is commonly deemed brain penetrant if the brain-to-plasma ratio is >0.04 using non-perfused brain tissue, as cerebral blood volume approximates 400 of total brain volume. In this study, the brain-to-plasma ratio, calculated using AUC or % ID at 24 hours (1440 minutes), was 0.447 and 0.437, respectively. This is a positive result, especially when considering brains were perfused prior to quantification of radioactivity. In this study, the brain-to-plasma ratio was calculated after a single bolus dose of Deflexifol. The brain-to-plasma ratio may therefore vary considerably in steady state versus non-steady state conditions. The high brain-to-plasma ratio calculated in this study suggests that 5-FU administered as Deflexifol is able to effectively penetrate into the brain. The results may be particularly significant considering that the brains were perfused prior to quantification of radioactivity. A more precise representation of brain uptake/BBB permeability can be determined using pharmacodynamics or receptor occupancy in addition to pharmacokinetic measurement.

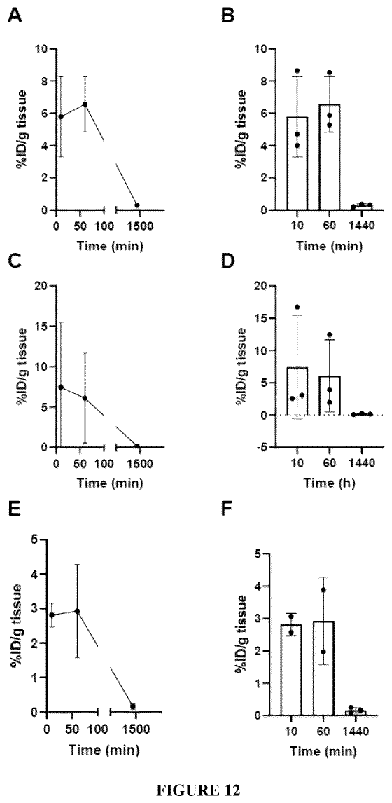

Clearance of Deflexifol spiked with 5 μCi 6-[³H]5FU was also assessed in the kidneys (FIG. 12, images A and B) and liver (FIG. 12, images C to F). For FIG. 12, images C and D show the original/unmodified data liver, and for images E and F, the outliers have been removed after applying ROUT Q=1%. All values are corrected to activity per gram of tissue and are presented as mean+/−SD percent of injected dose (n=2-3 per time point). Data analysed using Graphpad Prism v9.

The Cₘₐₓ in the kidney was 6.56% ID/g±1.73, Tₘₐₓ was 60 minutes and AUC was 5047 (std. error 1199). While the Cₘₐₓ in the liver was 7.45% ID/g±8.03, Tₘₐₓ was 10 minutes and AUC was (4656 (std. error 3852). Given the large variation in data observed for the liver at the 10 minutes and 60 minutes time points, ROUT was applied with an aggression of Q=1% to identify outliers and treated all values in the sub-column as one set of data. Such a test identified two outliers (FIG. 12, images E and F). After removing the outliers the adjusted Cₘₐₓ in the liver was 2.93% ID/g±1.35, Tₘₐₓ was 60 minutes and AUC was 2274 (std. error 934.4). A summary of the major pharmacokinetic parameters can be found in Table 9.

TABLE 9
A summary of the major pharmacokinetic parameters.

| | Plasma | Brain | Kidney | Liver* |
|---|---|---|---|---|
| T_{1/2} (minutes) | 22.18 | 44.16 | — | — |
| Kₑₗᵢₘ (min⁻¹) | 0.03 | 0.02 | — | — |
| Cₘₐₓ (% ID/ml | 100 | 1.26 ± 0.11 | 6.56 ± 1.73 | 7.45 ± 8.03 |
| or % ID/g) | | | | (2.93 ± 1.35) |
| Tₘₐₓ (minutes) | 0 | 10 | 60 | 10 (60) |
| AUC₁₀₋₁₄₄₀ | 1357 ± 334.2 | 606.7 ± 143.3 | 5047 ± 1199 | 4656 ± 3852 |
| | | | | (2274 ± 934.4) |

Values shown are mean ± SD; T_{1/2}—elimination half-life; Kₑₗᵢₘ—elimination rate constant; AUC—area under the curve; Tₘₐₓ—the time required to reach Cₘₐₓ = 0 for intravenous administration in plasma; Cₘₐₓ—maximum plasma concentration.
*parentheses indicate modified data with outliers removed.

### Study Conclusions

This study confirmed the uptake of 6-[³H]5FU into the brains of juvenile mice when administered in the Deflexifol formulation. The data indicates 5-FU, when delivered as Deflexifol, rapidly crosses the BBB and is cleared quickly, with less than 0.15% of the injected dose per gram of tissue remaining in the brain 24 hours post-intravenous administration.

### Example 6—Deflexifol Effect on U-87-MG Human Glioblastoma Cell Line

### Cell Lines Used

U-87-MG cells are maintained in DMEM (high glucose)+10% FCS

### Tested Drugs

Deflexifol (5-FU:Leucovorin ratio=15:1 mg/115 mM:1.95 mM) with: 5-FU concentrations: 120 nM, 470 nM, 1.9 μM, 7.5 μM, 30 μM, 120 μM, 479 μM, 1917 μM and 7600 μM; and equal Leucovorin calcium concentrations of: 1.9 nM, 7.9 nM, 127 nM, 507 nM, 2.03 μM, 8.125 μM, 32.5 μM and 130 μM.
5-FU single agent with concentrations of: 120 nM, 470 nM, 1.9 μM, 7.5 μM, 30 μM, 120 μM, 479 μM, 1917 μM and 7600 μM

### Experimental Setup

96-well 2D format (triplicate wells per drug concentration).
Cells seeded at 10,000 cells/well>24 hours incubation.
Drugs added 24 hours later and cells incubated a further 72 hours.
Cells imaged every 6 hours using Incucyte ZOOM for cell proliferation (confluency measurement) and evidence of cell death.

### Results

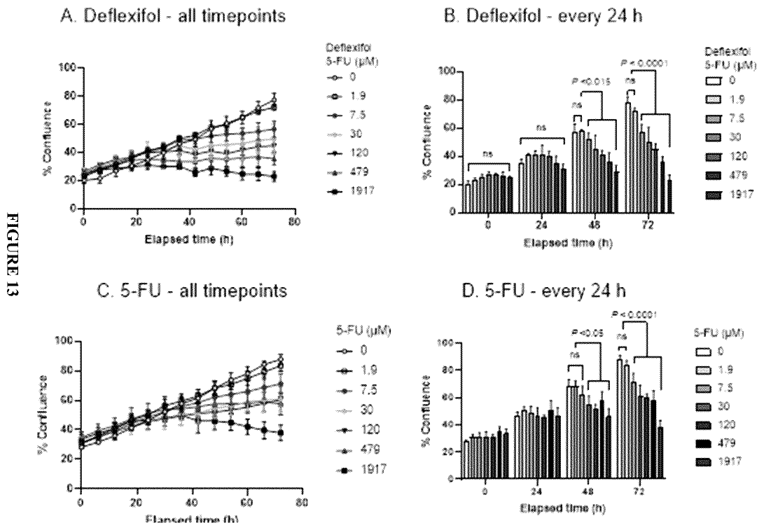

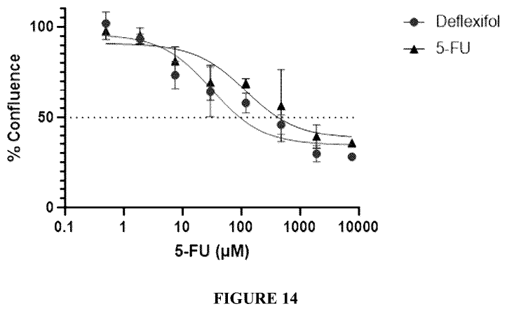

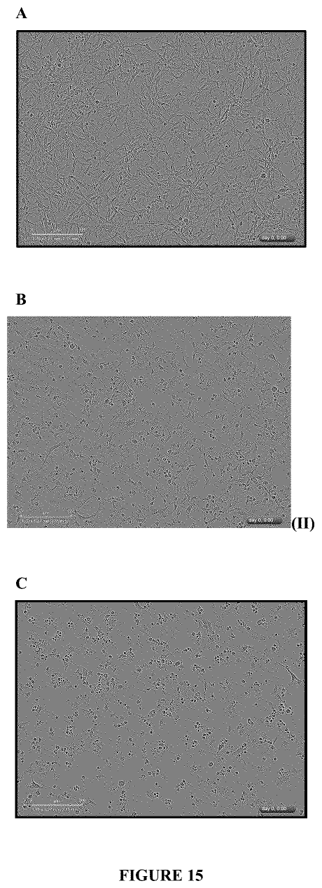

The results are shown in FIG. 13. 5-FU in Deflexifol or alone inhibits cell proliferation (FIG. 13, images A-D). Deflexifol (IC₅₀ 32 μM) is more effective than 5-FU alone (IC₅₀ 120 μM) on inhibiting cell proliferation (FIG. 14). In addition, Deflexifol causes cell death at very high concentration (>500 μM) (FIG. 15). FIG. 15 shows evidence of cell death with Deflexifol with:
(A) Cells after 72 hours culture no drug.
(B) Cells after 72 hours culture with 480 μM 5-FU (in DF).
(C) Cells after 72 hours culture with 7 mM 5-FU (in DF).

### Example 7—Clinical Study of Paediatric Ependymoma

The objective of this study is to determine efficacy of the composition used in the methods of the present disclosure in human paediatric ependymoma subjects.

Patients under the age of 18 (or 1 to 21 years of age) suffering from paediatric ependymoma can be treated in approved clinical studies involving administering the composition as described herein. The composition can be provided at a 5-FU to FA (Leucovorin) ratio of 15:1, termed ‘Deflexifol’. The composition can be administered in doses determined to be non-toxic in Examples 2-3

The absence/presence of improvement of condition and any side effects can be evaluated by clinical observations. The results can be analysed on an ongoing basis to determine whether the dose needs to be adjusted and/or the therapy should be administered in combination with any of the additional described therapies such as chemo- and/or radiotherapy.

### Example 8—Clinical Study of Adult Glioblastoma

The objective of this study is to determine efficacy of the composition used in the methods of the present disclosure in human adult glioblastoma subjects.

Patients above the age of 18 suffering from glioblastoma can be treated in approved clinical studies involving administering the composition as described herein. The composition can be provided at a 5-FU to FA (Leucovorin) ratio of 15:1, termed ‘Deflexifol’. The composition can be administered in doses determined to be non-toxic in Examples 2-3

The absence/presence of improvement of condition and any side effects can be evaluated by clinical observations. The results can be analysed on an ongoing basis to determine whether the dose needs to be adjusted and/or the therapy should be administered in combination with any of the additional described therapies such as chemo- and/or radiotherapy.

It will be appreciated by persons skilled in the art that numerous variations and/or modifications may be made to the above-described embodiments, without departing from the broad general scope of the present disclosure. The present embodiments are, therefore, to be considered in all respects as illustrative and not restrictive.

1. A method of treating and/or preventing a cancer of the brain and/or central nervous system in a subject in need thereof, the method comprising administering to the subject a composition comprising:
(i) 5-fluorouracil (5-FU), a pharmaceutically acceptable salt thereof, or a pharmaceutically acceptable analogue thereof;
(ii) folinic acid, or a pharmaceutically acceptable salt thereof; and
(iii) a cyclodextrin, or a pharmaceutically acceptable salt thereof.

2. (canceled)

3. (canceled)

4. The method according to claim 1, wherein the cancer is selected from: glioma, glioblastoma, oligodendroglioma, primitive neuroectodermal tumour, low, mid or high grade astrocytoma, ependymoma, oligodendroglioma, medulloblastoma, gliosarcoma, meningioma, pituitary carcinoma, neuroblastoma, craniopharyngioma, or one or more secondary metastases.

5. The method according to claim 1, wherein the cancer is a glioblastoma multiforme (GBM).

6. The method according to claim 1, wherein the cancer is an ependymoma.

7. The method according to claim 6, wherein the ependymoma is selected from a myxopapillary ependymoma, papillary ependymoma, subependymoma, or anaplastic ependymoma.

8. The method according to claim 6, wherein:
the ependymoma is a paediatric ependymoma or
the ependymoma is an adult ependymoma.

9. The method according to the claim 1, wherein the cancer is recurrent or has one or more residual primary lesions after previous treatment.

10. (canceled)

11. The method according to claim 1, wherein
the cyclodextrin or salt thereof is present at a concentration of from 10 mg/ml to 300 mg/ml;
the 5-FU, or a pharmaceutically acceptable salt or an analogue thereof, is present at a concentration of from 5 mg/ml to 50 mg/ml;
the folinic acid or salt thereof is present at a concentration of from 1 mg/ml to 15 mg/ml;
the cyclodextrin or pharmaceutically acceptable salt thereof to the 5-FU, pharmaceutically acceptable salt or an analogue thereof, is between 1:10 and 3:1; and/or
the pharmaceutically acceptable analogue of 5-FU is 5-fluoro-2-deoxyuridine (5-FUdr).

12. (canceled)

13. (canceled)

14. (canceled)

15. (canceled)

16. The method according to claim 1, wherein the composition is in the form of an aqueous solution.

17. (canceled)

18. The method according to claim 1, wherein the cyclodextrin is selected from; a hydroxyalkyl cyclodextrin, a sulfated cyclodextrin, a sulfoalkylether cyclodextrin, or a salt thereof.

19. The method according to claim 1, wherein the cyclodextrin is;
a β-cyclodextrin, or a salt thereof;
a hydroxypropyl β-cyclodextrin or a salt thereof;
2-hydroxypropyl β-cyclodextrin or a salt thereof;
a β-cyclodextrin sulfated salt;
a polysulfated β-cyclodextrin or a salt thereof; or
a heptakis(6-O-sulfo)-β-cyclodextrin salt.

20. (canceled)

21. (canceled)

22. The method according to claim 1, wherein the composition is administered intravenously or subcutaneously, or is administered as an intravenous infusion, as a bolus intravenous injection or infusion, and/or as a bolus intravenous injection or infusion, followed by an intravenous infusion.

23. (canceled)

24. The method according to claim 1, wherein the composition is administered at a dose of:
from 450 mg/m² to 3000 mg/m²; or
from 450 mg/m² to 3400 mg/m².

25. The method according to claim 1, wherein: the subject is also;
administered an effective amount of one or more other chemotherapeutic agents as part of a drug regimen: or administered an effective amount of one or more other chemotherapeutic agents as part of a drug regimen in combination with radiotherapy.

26. The method according to claim 25, wherein the one or more chemotherapeutic agents is selected from: temozolomide, dacarbazine, carmustine, lomustine, paclitaxel, docetaxel, vincristine, vinblastine, vinorelbine, etoposide, teniposide, topotecan, irinotecan, doxorubicin, daunomycin, epirubicin, idarubicin, methotrexate, cytarabine, gemcitibine, capecitabine, cisplatin, carboplatin, cyclophosphamide, oxaliplatin, or a mixture thereof.

27. (canceled)

28. The method according to claim 1, wherein:
the subject is also undergoing radiotherapy; or
the composition is formulated for a subject undergoing radiotherapy.

29. (canceled)

30. The method according to claim 1, wherein: the subject is a paediatric subjects.

31. (canceled)

32. A method of reducing the size of, or slowing the growth of, brain neoplasms in a subject in need of such treatment, the method comprising administering to the subject an effective amount of a composition comprising:
(i) 5-fluorouracil (5-FU) or an analogue or a pharmaceutically acceptable salt thereof;
(ii) folinic acid, or a pharmaceutically acceptable salt thereof; and
(iii) a cyclodextrin, or a pharmaceutically acceptable salt thereof.

33. The method according to claim 1, wherein the composition is formulated for administration with an effective amount of one or more other chemotherapeutic agents.

34. The method of claim 33, wherein:
the one or more other chemotherapeutic agents comprises an EGFR inhibitor;
the one or more other chemotherapeutic agents comprises an immunomodulatory agent; or
the one or more other chemotherapeutic agents comprises Atezolizumab, Avelumab Ipilimumab, Bevacizumab, Cemiplimab, Durvalumab, Pembrolizumab, Nivolumab and/or Pidilizumab.
